(19) United States
(12) Patent Application Publication
(10) Pub. No.: US 2024/0261366 A1
(43) Pub. Date: Aug. 8, 2024

(54) TREATMENT OF BETA-CELL FAILURE OR DYSFUNCTION

(71) Applicants: NOVMETAPHARMA CO., LTD., Seoul (KR); KOREA ADVANCED INSTITUTE OF SCIENCE AND TECHNOLOGY, Daejeon (KR)

(72) Inventors: Hail KIM, Daejeon (KR); Hoeyune JUNG, Pohang-si (KR); Kyun Hoo KIM, Daejeon (KR); Joonyub LEE, Seoul (KR); Hyeonkyu LEE, Daejeon (KR); Heon Jong LEE, Incheon (KR)

(73) Assignees: NOVMETAPHARMA CO., LTD., Seoul (KR); KOREA ADVANCED INSTITUTE OF SCIENCE AND TECHNOLOGY, Daejeon (KR)

(21) Appl. No.: 18/427,249

(22) Filed: Jan. 30, 2024

Related U.S. Application Data
(60) Provisional application No. 63/482,357, filed on Jan. 31, 2023.

(51) Int. Cl. A61K 38/12; A61K 33/30; A61K 45/06; A61P 1/18

(52) U.S. Cl. CPC A61K 38/12; A61K 33/30; A61K 45/06; A61P 1/18

(57) ABSTRACT

A method and composition for treating and/or preventing β-cell dysfunction and/or protecting impaired β-cells are disclosed. The method includes administering an effective amount of cyclo (his-pro) (CHP), alone or with zinc, to a subject. The composition contains an effective amount of cyclo (his-pro) (CHP) as an active ingredient. The composition may further contain zinc and/or other antidiabetic agent, or can be administered in combination with zinc and/or other antidiabetic agent.

### CROSS REFERENCE TO RELATED APPLICATIONS

This application claims benefits of and priority based on U.S. Provisional Application No. 63/482,357 filed Jan. 31, 2023, of which the entire content is incorporated by reference.

### TECHNICAL FIELD

The present disclosure generally relates to the field of β-cell mediated disorders, impaired β-cells, and/or β-cell failure. In particular, the present disclosure relates to cyclo (his-pro) (CHP) for use in the treatment or prevention of disorders and/or symptoms in association with impaired β-cells or β-cell failure. The present disclosure also relates to CHP for use in regenerate functional β-cells.

### BACKGROUND

The pancreas is both an endocrine and exocrine gland. In its function as an endocrine gland, it produces several hormones, including insulin, glucagon, somatostatin and pancreatic polypeptide. Insulin and glucagon are secreted from beta and alpha cells, respectively, to regulate carbohydrate, protein and lipid metabolism. The exocrine pancreas includes ductal and acinar cells, which synthesize and secrete digestive enzymes that aid in the digestion of food. Diseases and disorders associated with the pancreas include diabetes, exocrine pancreatic insufficiency, and the like.

Overt diabetes develops when the pancreatic β-cells cannot satisfy the demand of insulin that is required to maintain a normal glucose metabolism. The state that functional β-cell mass, which is the sum of the number of β-cells and functional state of each β-cell, is markedly decreased to a level that is insufficient to sustain a normal glucose metabolism, is referred to as “β-cell failure.” Preservation of insulin-producing β-cell mass and function is key to attenuate disease progression in all forms of diabetes.

It has been reported that β-cell failure begins long before the onset of overt diabetes and progresses over a long period of time. Recently, as the dematuration phenomenon in which β-cell identity is lost due to the persistence of β-cell failure is considered as one of the main mechanisms of diabetes.

For example, in prediabetic people, chronic glucose intolerance and elevated blood glucose levels continuously exacerbate β-cell workload and stress, culminating in cellular exhaustion, cell death, and clinical manifestation of hyperglycemia. Thereafter, uncontrolled hyperglycemia, often in concert with other cytotoxic factors, leads to accelerated β-cell mass loss and functional deterioration in overt diabetic patients. Therefore, the progressive β-cell failure (in mass and function) during the prediabetes phase is the major factor responsible for the occurrence of type 2 diabetes. Nonetheless, the precise contribution of β-cell mass and function to the pathogenesis of diabetes as well as the underlying mechanisms are still unclear. For example, β-cell function is already decreased by 50% in subjects with fasting glucose or 2 hour plasma glucose levels at the upper limit of the normal range (respectively 95-100 mg/dL, and 130-139 mg/dL) whereas subjects diagnosed as type 2 diabetes have already lost over 80% of their β-cell function.

Currently, most clinical treatments of type 2 diabetes either target insulin resistance or elevate insulin levels. In light of a potential exhaustive effect on β-cells, a therapeutic strategy that can prevent β-cell failure and restore β-cell function by regenerating β-cell mass or by preserving β-cell function is needed.

Dietary and lifestyle changes, including healthier dietary habits and increased exercise, can be very efficient in preventing or treating prediabetes, type 2 diabetes and/or gestational diabetes mellitus. However, patient compliance is often an issue. And, antidiabetic drugs such as biguanides and thiazolidinediones may also be used. However, many of these have undesirable side effects. Moreover, there is no practice of giving such drugs to prevent prediabetes and many are unsuitable for use during pregnancy. Accordingly, there is a need to find alternative ways to treat or prevent prediabetes, type 2 diabetes, gestational diabetes mellitus and/or a condition associated with impaired or failed β-cell function in a subject.

Cyclo (His-Pro) (CHP) is a cyclic dipeptide and shows a wide range of biological activities, which include anti-inflammation effects by modulating the Nrf2 and Nf-Kb signaling and hypoglycemic activities by influencing the expression of various proteins involved in glucose homeostasis such as apolipoproteins and fibrinogen. These biological effects render CHP a potential therapeutic agent in neuro-degenerative diseases. A treatment using CHP in combination with zinc and histidine on glucose metabolism in genetically obese type 2 diabetic mice was studied. Hwang et al. Diabetes Obes Metab. 2003 September; 5(5):317-24, doi: 10.1046/j.1463-1326.2003.00281.x. A combination of zinc, CHP, and arachidonic acid was also studied in streptozotocin-induced diabetic rats. Song et al., Metabolism. 2001 January; 50(1):53-9, doi: 10.1053/meta.2001.19427. Despite that some biological pathways have been clarified, the precise mechanisms are still debated.

It has been reported that CHP in combination with zinc can improve hyperglycemia and lipid metabolism by reducing lysine acetylation of peroxisome proliferator-activated receptor gamma coactivator 1-alpha, liver kinase B1, and nuclear factor-κB p65 in the liver and visceral adipose tissue in KK-Ay mice (a type 2 diabetes mellitus model) (Jeon et al., Diabetes & Metabolism Journal, 2023; 47: pp. 653-667) and that CHP in combination with zinc stimulated insulin degrading enzyme (IDE) synthesis, which might be applicable to clinical applications to treat Alzheimer's disease (AD) and type 2 diabetest mellitus (Song et al., BBA Clinical, 9 (2017), pp. 41-54).

However, direct protective and regeneration effects of functional β-cell mass by CHP have not been reported.

### SUMMARY

According to an aspect of the present disclosure, a method of regenerating functional (insulin secreting) β-cell mass comprising administering an effective amount of a cyclo (His-Pro) (CHP), a pharmaceutically acceptable salt thereof, a stereoisomer, or a solvate thereof to a subject in need of such treatment. In embodiments, the CHP may be administered in combination with zinc and/or other antidiabetic drug. In some embodiments, the subject has decreased insulin secreting β-cell mass by at least 10%, at least 20%, at least 30%, at least 40%, at least 50%, at least 60%, at least 70%, at least 80%, or at least 90%, compared to a healthy subject. In still another embodiments, the subject has normal range of fasting and/or 2-hour blood glucose level and has decreased insulin secreting β-cell mass by at least 10%, at least 20%, at least 30%, at least 40%, at least 50%, at least 60%, at least 70%, at least 80%, or at least 90%, compared to a normal functional β-cell mass of a healthy subject.

In another embodiment, the present disclosure provides a method of preventing and/or treating β-cell failure or comprising administering an effective amount of a CHP, a pharmaceutically acceptable salt thereof, a stereoisomer, or a solvate thereof to a mammal in need thereof. In embodiments, the CHP may be administered in combination with zinc and/or other antidiabetic drug. In some embodiments, the subject has decreased insulin secreting β-cell mass by at least 10%, at least 20%, at least 30%, at least 40%, at least 50%, at least 60%, at least 70%, at least 80%, or at least 90%, compared to a healthy subject. In still another embodiments, the subject has normal range of fasting and/or 2-hour blood glucose level and has decreased insulin secreting β-cell mass by at least 10%, at least 20%, at least 30%, at least 40%, at least 50%, at least 60%, at least 70%, at least 80%, or at least 90%, compared to a normal functional β-cell mass of a healthy subject.

Still another aspect of the present disclosure provides a method of preventing and/or treating β-cell mediated disorder linked to an impaired function of insulin secreting β-cells and/or a condition or symptom associated with such disorders, comprising administering an effective amount of a CHP, a pharmaceutically acceptable salt thereof, a stereoisomer, or a solvate thereof to a mammal in need thereof. In embodiments, the CHP may be administered in combination with zinc and/or other antidiabetic agent. In the embodiments, the symptom associated with an impaired β-cell function is selected from the group consisting of un-usual hunger, increased thirst, un-usual bed-wetting, un-usual mood changes, irritability, fatigue, frequent urination, blurred eye sight, un-intended weight-loss, overweightness, obesity, and combinations thereof. In other embodiments, the disorder associated with an impaired β-cell function is selected from the group consisting of nephropathy, heart disease, neuropathy, blood vessel disease, skin infections, complications during pregnancy, impaired vision due to damages in the blood vessels of the retina, foot complications, cardiovascular diseases, fatty liver diseases, and combinations thereof. In some embodiments, the subject has decreased insulin secreting β-cell mass by at least 10%, at least 20%, at least 30%, at least 40%, at least 50%, at least 60%, at least 70%, at least 80%, or at least 90%, compared to a healthy subject. In still another embodiments, the subject has normal range of fasting and/or 2-hour blood glucose level and has decreased insulin secreting β-cell mass by at least 10%, at least 20%, at least 30%, at least 40%, at least 50%, at least 60%, at least 70%, at least 80%, or at least 90%, compared to a normal functional β-cell mass of a healthy subject.

An aspect of the present disclosure also relates to CHP for use as a medicament for treating disorder associated with an impaired β-cell function. In embodiments, the medicament may further comprise zinc and/or other antidiabetic agent.

In some embodiments, the present disclosure further relates to CHP for use in the treatment or prevention of disorders linked to an impaired function of insulin secreting β-cells and/or a condition or symptom associated with such disorders. Another aspect of the present disclosure also relates to the use of CHP for the manufacture of a composition for the treatment or prevention of disorders linked to an impaired function of insulin secreting β-cells and/or a condition or symptom associated with such disorders. In embodiments, the CHP may be used together with zinc and/or another antidiabetic agent.

According to some aspects, the present disclosure is related to a method of preventing and/or treating diabetes or prediabetes comprising administering an effective amount of a blood glucose lowering agent and a CHP to a subject in need thereof. The method may further comprise administering zinc and/or another antidiabetic agent to the subject.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram showing the generation of an animal model and the experimental protocol.

FIG. 2 shows the results of body weight changes and body composition analysis. In FIG. 2, “Cyclo-Z” is a combination of CHP and zinc.

FIG. 3 shows the results of glucose tolerance test (GTT) and insulin tolerance test (ITT) performed in 12- and 26-week-old mice.

FIG. 4 shows immune-fluorescence staining images of expressions of genes involved in maintaining β-cells identity. In FIG. 4, the Prmt1 BiKO panel indicates a Prmt1 knocked out (KO) animal group, the “KO+Vehicle” panel indicates a Prmt1 knocked out (KO) animal group administered with vehicle alone, and “KO+Cyclo-Z” panel indicates a Prmt1 knocked out (KO) animal group administered with a combination of CHP+Z. The results show that the expressions of MAFA, UCN3, and SLC2A2 proteins in the KO+Cyclo-Z group are as high as the control group.

FIG. 5 shows immune-fluorescence staining images of expressions of genes indicating dematuration of β-cells. In FIG. 5, the Prmt1 BiKO panel indicates a Prmt1 knocked out (KO) animal group, the “KO+Vehicle” panel indicates a Prmt1 knocked out (KO) animal group administered with vehicle alone, and “KO+Cyclo-Z” panel indicates a Prmt1 knocked out (KO) animal group administered with a combination of CHP+Z. The results show that the expressions of ALDHIA3 and CCK proteins, which show dematuration of β cells, was significantly reduced in the Cyclo-Z administration group compared to the Prmt1 BiKO group and the KO+Vehicle group.

FIG. 6A shows immune-fluorescence staining images of proinsulin in β cells in PRMT1 BiKO mouse model fed with a high-fat diet. In FIG. 6, the Prmt1 BiKO panel indicates a Prmt1 knocked out (KO) animal group, the “KO+Vehicle” panel indicates a Prmt1 knocked out (KO) animal group administered with vehicle alone, and “KO+Cyclo-Z” panel indicates a Prmt1 knocked out (KO) animal group administered with a combination of CHP+Z. The results show that proinsulin and insulin levels are low in the KO+Cyclo-Z, whereas the Prmt1 BiKO group and the KO+Vehicle group had elevated levels of proinsulins and insulins compared to the control group.

FIG. 6B shows the proinsulin and C-peptide levels in plasma of the KO+Cyclo-Z group (Cycloz) and the KO+Vehicle group (Vehicle) in 26-weeks old Prmt1 BiKO mice.

FIG. 7 shows immune-fluorescence staining images of 8-oxo-dG level, an indicator of oxidative stress, in β cells in PRMT1 BiKO mouse model. The results show that the KO+Cyclo-Z group animals exhibit significantly reduced oxidative stress.

FIG. 8 shows the transcript analysis of the effect of Cyclo-Z at the transcriptome level using single-cell RNA-sequencing analysis technique.

FIG. 9 are transmission electron microscopic (TEM) images of the β-cells of PRMT1 BiKO mice administered with vehicle (PRMT1 KO (Vehicle) and PRMT1 BiKO mice administered with Cyclo-Z (PRMT1 KO (Cyclo-Z)). In FIG. 9, Red: immature insulin granule, Yellow: endoplasmic reticulum, Blue: autophagosome, Green: mitochondria. The TEM images of FIG. 9 show the protective effect of β-cell organelles by Cyclo-Z. Compared to the vehicle administered animal group, the density of insulin granules increased in the Cyclo-Z administered animal group, and dense core granules and halo structures, which are characteristics of mature insulin granules, were observed in the Cyclo-Z administered animal group.

FIGS. 10A-10Q show the effects of Prmt1 deletion in beta cell identity changes observed in inducible beta-cell-specific Prmt1 knockout mouse model.

FIGS. 11A-11H show that proinsulin processing is impaired in Prmt1BiKO islets.

FIGS. 12A-12P show that a long-term high fat diet (HFD) exacerbates beta cell dedifferentiation in Prmt1BiKO animal model.

FIGS. 13A-13N show that CHP in combination with zinc (CycloZ) prevents or suppresses beta cell dysfunction in Prmt1BiKO animal model.

FIGS. 14A-14O show that CHP in combination with zinc (CycloZ) protects beta cell from identity loss and promotes regeneration of impaired beta cells.

### DETAILED DISCLOSURE

### Definitions

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present disclosure, particular methods and materials are now described.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein. In an aspect, the word “about” as used in referring to a numerical value is intended to include 10%, 9%, 8%, 7%, 6%, 5%, 4%, 3%, 2%, or 1% variance from the numerical value(s).

The term “subject” as used herein refers to a mammal. The mammal may be an adult, child or infant.

The term “animal” used here includes all members of the animal kingdom including a human, a cat, a dog and the like. Thus, the term “mammal” includes both human and non-human mammals. Similarly, the term “subject” and “patient” includes both human and veterinary subjects. The subject may be a female, for example, a female who is trying to get pregnant, who is pregnant, or who is lactating.

The term “active agent” or “active ingredient” or “drug” or “medicament,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The term “Cyclo-Z” or “CycloZ” used in the Examples and the figures of this disclosure refers to a combination of CHP and zinc. Throughout the disclosure, the term “zinc” refers to a zinc element as well as a zinc compound as described herein, unless specifically identified otherwise.

The phrase “consists essentially of” used herein with regard to a composition or formulation means that the composition or formulation contains the listed compound(s) as sole active ingredient(s) and may additionally contain a pharmaceutically acceptable inert additive(s), excipient(s), or carrier(s). Such inert additives, excipients, or carriers are known in the art.

The terms “parenteral administration” and “administered parenterally” are art-recognized and refer to modes of administration other than enteral and topical administration, usually by rol intake, injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection.

The term “treatment or treating” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. It may for example encompass the reduction of the severity of a disorder in a subject.

The phrase “pharmaceutically acceptable” additives, excipients, or carriers as used herein include those well known in the art. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “pharmaceutically acceptable salt” refers to a salt that can be pharmaceutically used, among the substances having cations and anions coupled by electrostatic attraction. Typically, it may include metal salts, salts with organic bases, salts with inorganic acids, salts with organic acids, salts with basic or acidic amino acids or the like. Examples of the metal salts may include alkali metal salts (sodium salts, potassium salts, etc.), alkaline earth metal salts (calcium salts, magnesium salts, barium salts, etc.), aluminum salts or the like; examples of the salts with organic bases may include salts with triethylamine, pyridine, picoline, 2,6-lutidine, ethanolamine, diethanolamine, triethanolamine, cyclohexylamine, dicyclohexylamine, N,N′-dibenzylethylenediamine or the like; examples of the salts with inorganic acids may include salts with hydrochloric acid, hydrobromic acid, nitric acid, sulfuric acid, phosphoric acid, or the like; examples of the salts with organic acids may include salts with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid or the like; examples of the salts with basic amino acids may include salts with arginine, lysine, ornithine or the like; and examples of the salt with acidic amino acids include salts with aspartic acid, glutamic acid, or the like.

The term “therapeutically effective amount” or “effective amount,” or “effective dose,” as used herein, is the amount of the active agent(s) present in a composition described herein that is needed to provide an increase or restore or regenerate functional (insulin secreting) β-cell mass. The precise amount will depend upon numerous factors, for example the specific activity of the composition, the delivery device employed, the physical characteristics of the composition, its intended use, as well as patient considerations such as severity of the disease state, patient cooperation, etc.

The terms “increased” or “increase” or “prolong” are used herein to generally mean an increase or prolong by a statically significant amount; in some embodiments, the terms “increased” or “increase” or “prolong” mean an increase or prolong of at least 10% as compared to a reference level (e.g., without the treatment or the administration described herein), for example an increase of at least about 10%, at least about 20%, or at least about 30%, or at least about 40%, or at least about 50%, or at least about 60%, or at least about 70%, or at least about 80%, or at least about 90% or up to and including a 100% increase (or prolong) or any increase (or prolong) between 10-100% as compared to a reference level. Other examples of “increase” or “prolong” include an increase or prolong of at least 2-fold, at least 5-fold, at least 10-fold, at least 20-fold, at least 50-fold, at least 100-fold, at least 1000-fold or more as compared to a reference level.

The term “suppress” or “suppressed” are used herein generally to mean that a progress of a disease or development of symptom(s) is slowed or decreased, compared to the absence of an intervention described herein.

The terms, “decreased” or “decrease” are used herein generally to mean a decrease by a statistically significant amount. In some embodiments, “decreased” or “decrease” means a reduction by at least 5% or at least 10%, as compared to a reference level (e.g., without the treatment or the administration described herein), for example a decrease by at least about 10%, or at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 5-100% or 10-100% as compared to a reference level. In the context of a marker or symptom, by these terms is meant a statistically significant decrease in such level. The decrease can be, for example, at least 5%, at least 10%, at least 20%, at least 30%, at least 40% or more, and is preferably down to a level accepted as within the range of normal for an individual without a given disease.

All references to singular characteristics or limitations of the present invention shall include the corresponding plural characteristic or limitation, and vice versa, unless otherwise specified or clearly implied to the contrary by the context in which the reference is made.

### B-Cell Failure or Impaired B-Cell Function and Associated Disorder

Several symptoms are associated with an impaired β-cell function. These symptoms are well known to the person skilled in the art. The subject matter of the present disclosure may be used to treat or prevent such symptoms associated with an impaired β-cell function. In one embodiment of the present disclosure, the symptom associated with an impaired β-cell function may be selected from the group consisting of un-usual hunger, increased thirst, un-usual bed-wetting, un-usual mood changes, irritability, fatigue, frequent urination, blurred eye sight, un-intended weight-loss, overweightness, obesity, or combinations thereof.

Numerous conditions are associated with disorders linked to an impaired β-cell function. Such conditions are well known to the person skilled in the art. The subject matter of the present disclosure may be used to treat or prevent such conditions associated with disorders linked to an impaired β-cell function. In one embodiment of the present disclosure the condition associated with disorders linked to an impaired β-cell function may be selected from the group consisting of nephropathy, heart disease, neuropathy, blood vessel disease, skin infections, complications during pregnancy, impaired vision due to damages in the blood vessels of the retina, foot complications, cardiovascular diseases, fatty liver diseases, or combinations thereof.

Numerous conditions are associated with prediabetes, type 2 diabetes and/or gestational diabetes linked to an impaired β-cell function. In an embodiment of the disclosure the condition associated with gestational diabetes mellitus linked to an impaired β-cell function is selected from the group consisting of preterm and caesarean delivery, birth injury to the mother or baby, shoulder dystocia, macrosomia, excessive offspring blood glucose concentration, excess weight/adiposity and associated metabolic disorders e.g. type 2 diabetes, fatty liver disease and obesity immediately after birth and later in the life of the offspring, and an increased risk for the mother of having or developing type 2 diabetes immediately after birth and/or later in life.

In an embodiment of the disclosure, the subject e.g. the human subject, is at risk of suffering from pre-diabetes, type 2 diabetes or gestational diabetes mellitus, who usually do not have a favorable metabolic profile and/or have a history or family history of one or more of these disorders.

Gestational diabetes mellitus is a disorder that affects pregnant woman. Accordingly, in another embodiment of the present disclosure the disorder linked to an impaired function of functional β-cells is gestational diabetes mellitus or a condition associated therewith and the subject is a woman who is trying to get pregnant, is pregnant or who is lactating or is her offspring.

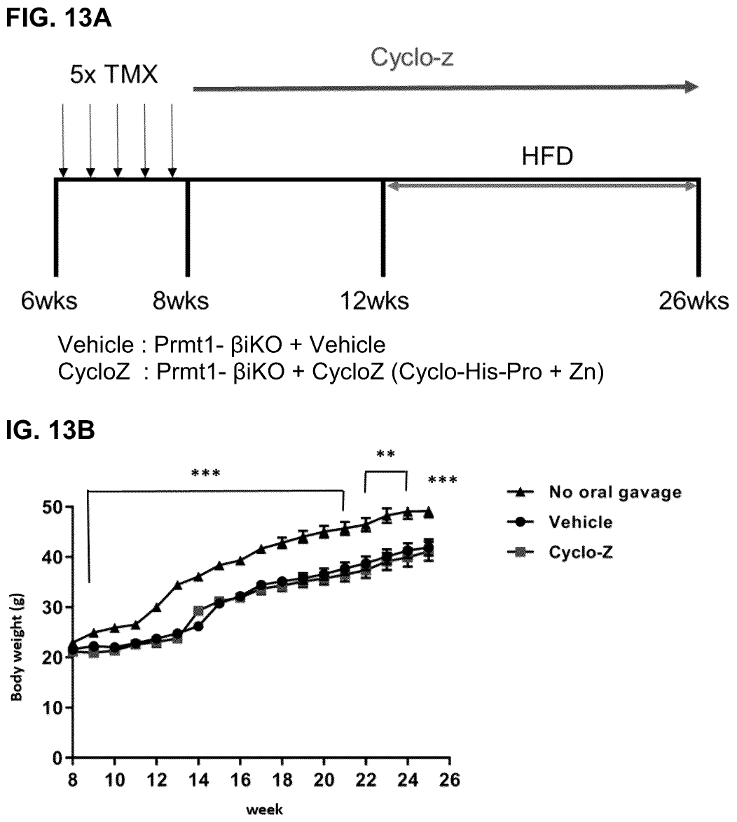

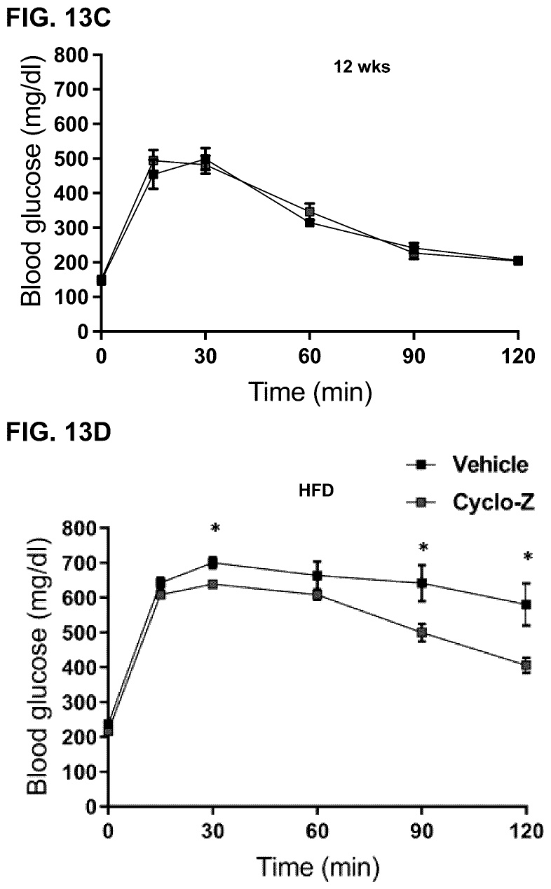

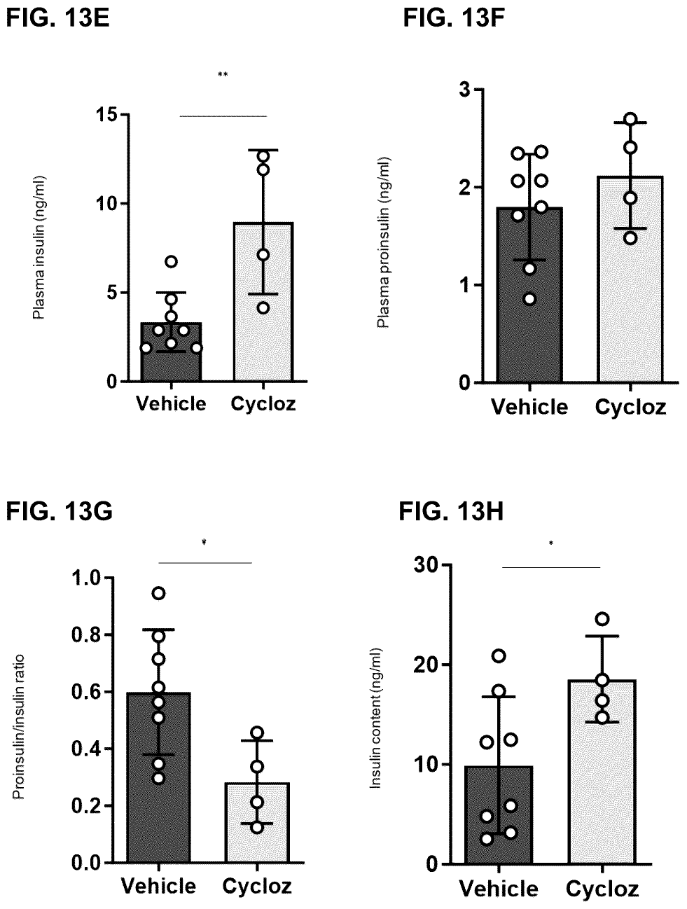

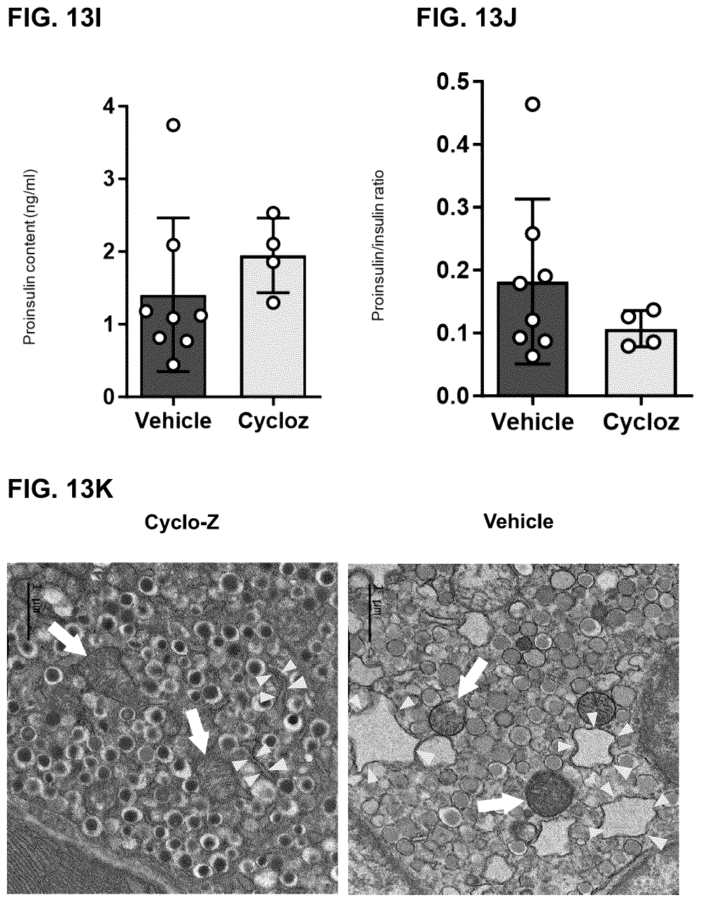

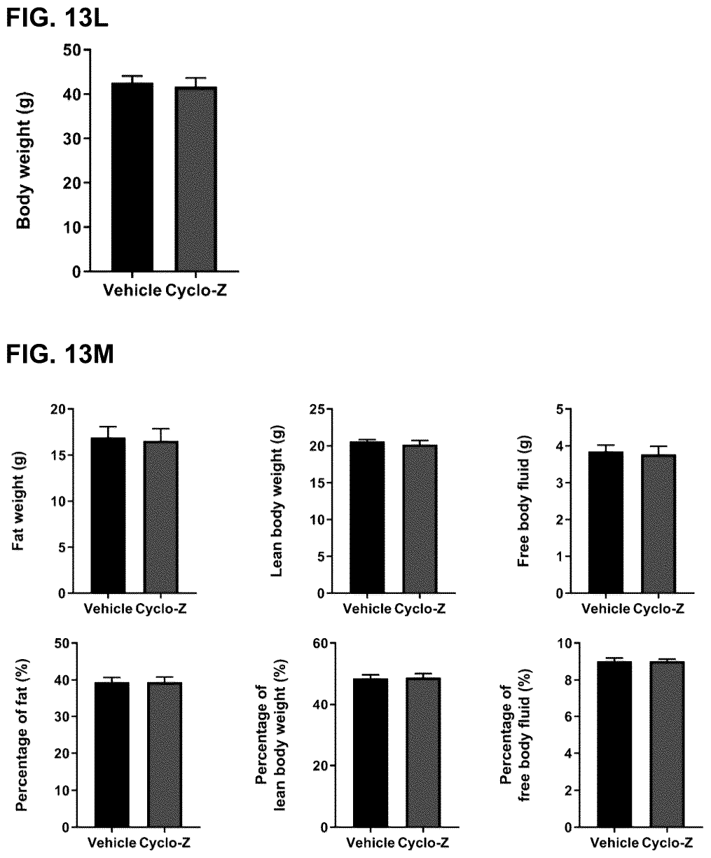

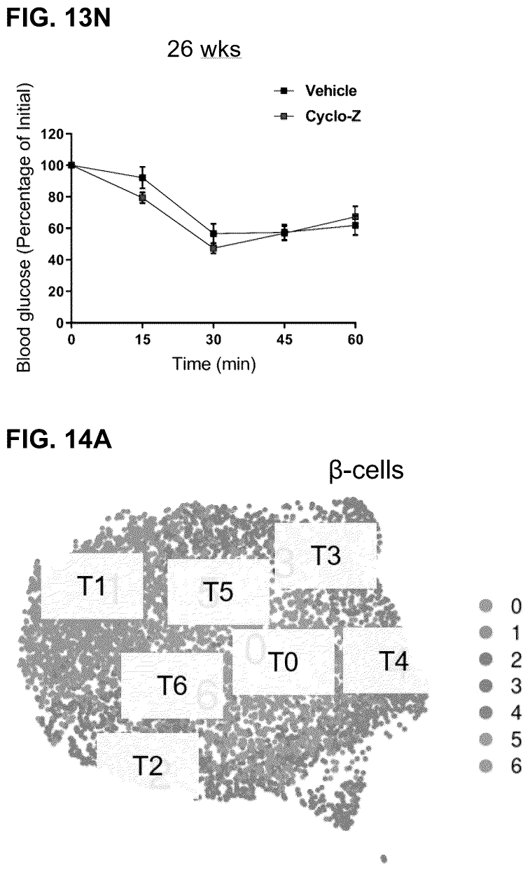

As evidenced by the experimental results disclosed herein, CHP or CycloZ administration increases plasma insulin level and insulin content in whole pancreas (FIGS. 13E and 13H), while plasma proinsulin level and proinsulin content showed non-significant changes (FIGS. 13F and 13I), and significantly increase plasma proinsulin/insulin ratio (FIG. 13G). Accordingly, in an embodiment of the disclosure, a method for increasing plasma insulin level and insulin content in whole pancreas and/or increase plasma proinsulin/insulin ratio in a subject in need thereof is disclosed, which comprises administering an effective amount of CHP, optionally in combination with zinc to a subject in need thereof. The subject may have impaired beta cell function.

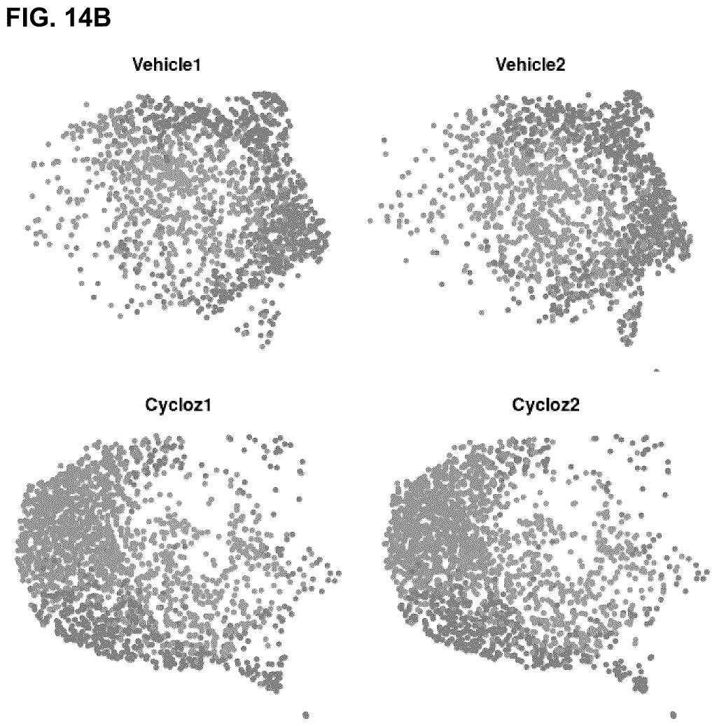

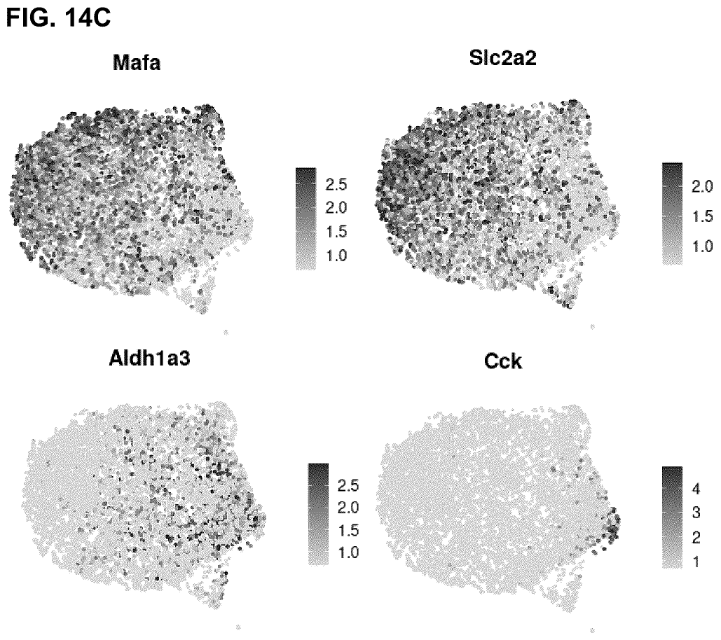

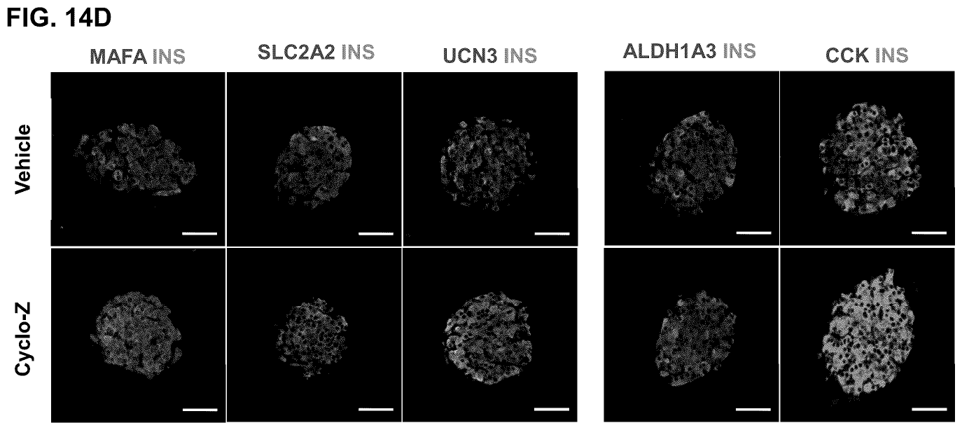

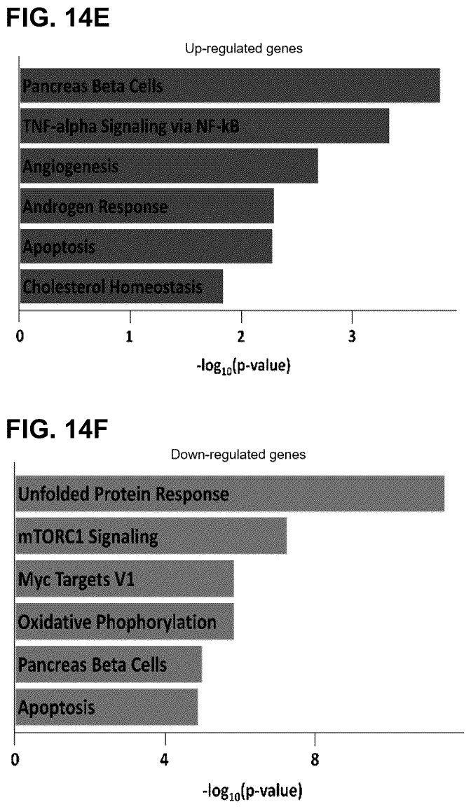

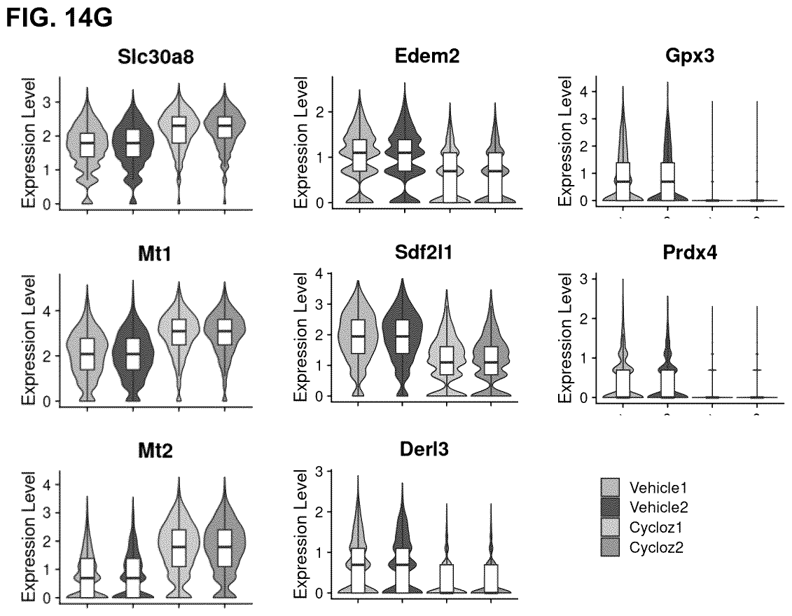

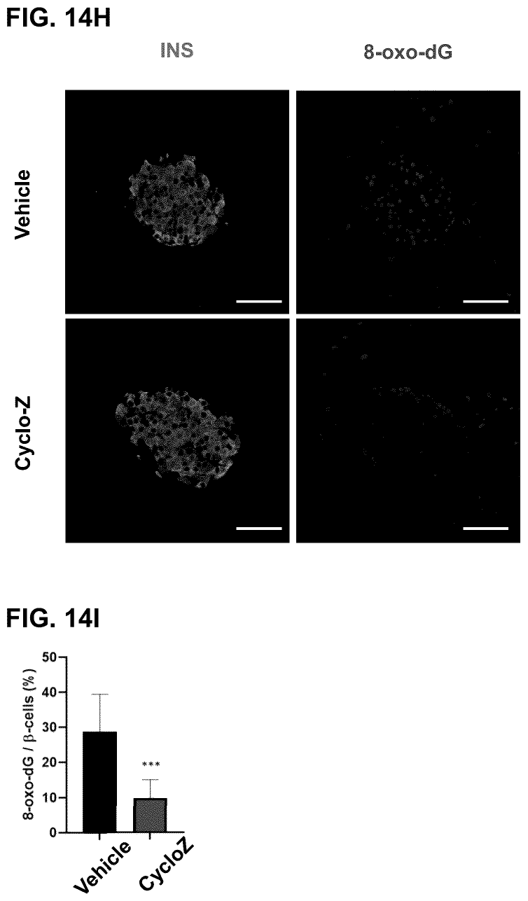

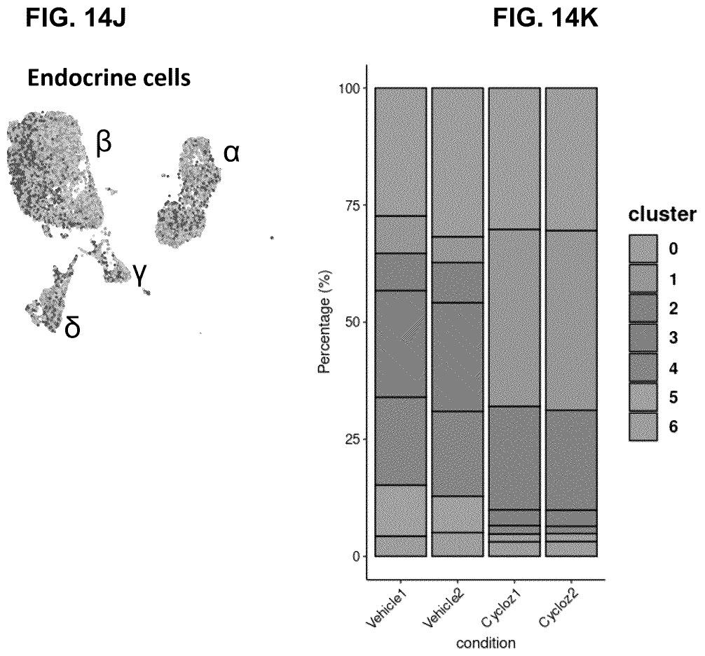

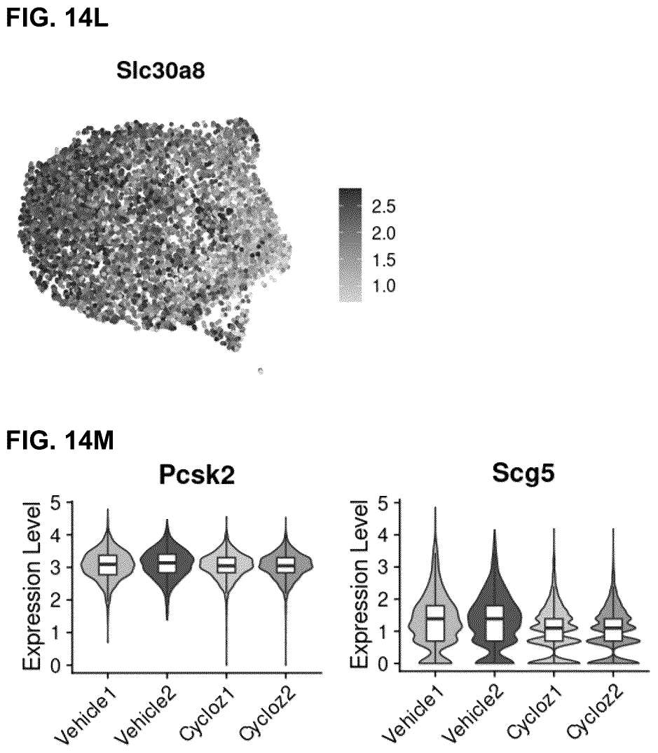

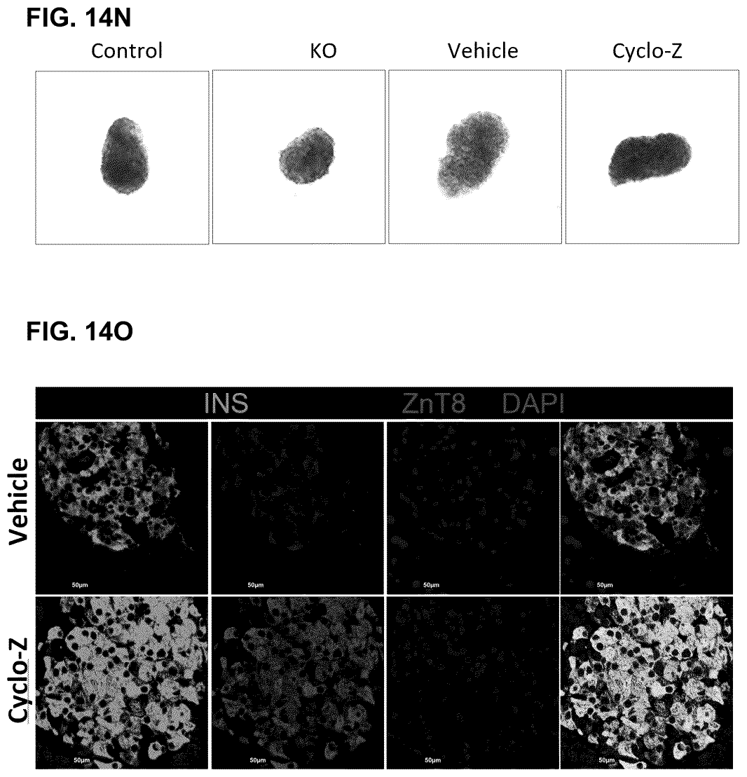

The endoplasmic reticulum (ER) of beta cells is the major organelle responsible for secretary protein (e.g., proinsulin, insulin, and the like) synthesis, folding and quality control. To maintain ER homeostasis during the stresses associated with secretory protein synthesis and folding, cells activate the intracellular transduction pathway termed the unfolded protein response (UPR). Recent studies suggest that animal models support that it is a dysregulated ER stress response that is more likely to predispose to diabetes onset, suggesting that a functional, homeostatic ER stress response is generally beta-cell protective in vivo. Therefore, UPR function is required for normal beta-cell health and survival. Accordingly, the results of relief or alleviation of ER-stress generated in HFD-fed Prmt1 BiKO islets and prevention of oxidative stress, by CHP or CycloZ treatment, suggest that the CHP or CycloZ protects beta cells from beta cell dysfunction. See FIGS. 14F-14I. If the CHP is to be administered to a human subject desiring to get pregnant, it may be to be administered during at least 1, 2, 3 or 4 months preceding the pregnancy or desired pregnancy. If the CHP is to be administered to a pregnant subject, it may be administered throughout or partially throughout the pregnancy e.g. for at least 4, at least 8, at least 12, at least 16, at least 20, at least 24, at least 28, or at least 36 weeks depending on the gestational period of the subject. Administration may also continue throughout or partially throughout the lactation period of said subject.

Since the risk of gestational diabetes mellitus increases in the second and third trimester of pregnancy, administration may be beneficial in the second and third trimester of pregnancy for the prevention or treatment of gestational diabetes mellitus linked to an impaired function of functional β-cells, or the prevention of a condition associated therewith in a pregnant subject or its offspring.

In an embodiment of the disclosure, the CHP is to be administered in at least the second and/or third trimester of pregnancy wherein the subject is a pregnant woman or her offspring.

In the methods, uses, and compositions described above, an additional substance such as zinc and/or another antidiabetic drugs may be used in combination with the CHP.

### CHP and Compositions Containing CHP

Cyclo (His-Pro) (CHP) is a naturally-generating cyclic dipeptide that is structurally related to thyrotropin-releasing hormone (TRH). The CHP is a peptide inherent in animal and human tissues and body fluids. The CHP is found in blood, semen, gastrointestinal tract, urine, etc., and in particular is a metabolite rich in prostate. The CHP has been known to have a variety of physiological functions such as anti-diabetes, anti-obesity, anti-inflammatory and antioxidant effects.

According to an aspect of the disclosure, CHP or a pharmaceutically acceptable salt thereof, a stereoisomer, or a solvate thereof is used as an active agent or active ingredient for the methods and compositions.

CHP is represented by the following formula:

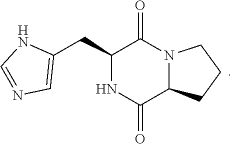 [SMILES: O=C1N[C@@H](Cc2cnc[nH]2)C(=O)N2CCC[C@@H]12]

According to the embodiments described herein, the CHP is used to broadly include CHP of the formulas above, a pharmaceutically acceptable salt thereof, a stereoisomer, a solvate thereof, unless specified otherwise.

As a non-limiting example of a CHP solvate, a CHP monohydrate is illustrated below:

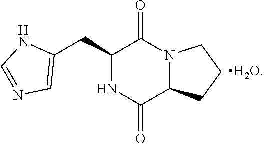 [SMILES: O=C1N[C@@H](Cc2cnc[nH]2)C(=O)N2CCC[C@@H]12]

In one embodiment, the CHP is substantially pure.

In an embodiment, the CHP is a CHP hydrate. In still another embodiment, the CHP hydrate is characterized by an XRPD diffractogram comprising peaks at about 17±0.2° and about 27.3±0.2° in 20. One embodiment of substantially pure CHP hydrate is characterized by an X-ray powder diffractogram comprising at least three peaks chosen from the following list: 13.7, 17, 18.1, 20.2, and 27.3 degrees (±0.2° in 20). Another embodiment is characterized by an XRPD diffractogram comprising at least two peaks chosen from the following list: 10, 13.7, 17, 18.1, 20.2, and 27.3 degrees (±0.2° in 20). CHP hydrate, as one of a CHP solvate, can be made by a process described in U.S. application Ser. No. 16/448,083, of which content is incorporated herein by reference, in its entirety.

CHP synthesized from different biochemical sources, including histidine-proline-rich glycoprotein. High levels of CHP are present in many food sources, and are readily absorbed in the gut without chemical or enzymatic destruction.

In one embodiment, a CHP may be present in a composition in amount ranging from about 0.5 to about 10000 mg, from about 1 to 5000 mg, from about 1 to 2000 mg, or from about 10 to about 1000 mg. In another embodiment, the amount of CHP present in the administered pharmaceutical composition may range from about 5 to about 3000 mg, from about 50 to about 2000 mg, from about 100 to about 2000 mg, from about 50 to about 1000 mg, from about 100 to about 1000 mg, from about 150 to about 2000 mg, from about 200 to about 1000 mg, from about 50 to about 800 mg, from about 100 to about 700 mg, from about 50 to about 600 mg, or from about 100 to about 1500 mg, as calculated in term of anhydrous CHP. The composition may be a pharmaceutical composition, foodstuff, or a dietary supplement. In particular embodiment, the composition is a pharmaceutical composition.

In another embodiment, the composition is suitable for prevention and/or treatment of a disorder associated with impaired β-cell function or β-cell failure in a subject. In still another embodiment, the composition may further comprise zinc and/or a known other blood glucose-lowering agent. Alternatively, CHP, zinc, and a blood glucose-lowering agent may be administered in separate formulations, simultaneously or sequentially. In another embodiment, the composition may consist essentially of CHP as an active ingredient. In still another embodiments, the composition may comprise CHP and/or zinc and/or other glucose-lowering agent. In some embodiments, the composition which comprises CHP, zinc, and/or other blood glucose-lowering agent may be administered or used separately from another composition comprising an another therapeutically active agent.

In some embodiments, the another therapeutically active agent may include a biomolecule, bioactive agent, small molecule, drug, prodrug, drug derivative, protein, peptide, vaccine, adjuvant, imaging agent (e.g., a fluorescent moiety), polynucleotide or a metal. Such drug includes an antidiabetic agent. In yet another embodiment, the active agent is a metal element, metal cation, a metal complex, or a metal compound wherein the metal can be copper, zinc, magnesium, manganese, iron, cobalt, chromium, or a combination thereof. In an embodiment, the metal is zinc and a zinc compound may be zinc gluconate, zinc acetate, zinc sulfate, zinc picolinate, zinc orotate, or zinc citrate. In another embodiment, the metal is magnesium and a magnesium compound such as magnesium oxide, magnesium citrate, magnesium chloride, magnesium glycinate, magnesium biglycinate, magnesium aspartate, magnesium lactate, or magnesium chloride can be employed. In another embodiment, the metal is manganese and a manganese compound may include manganese amino acid chelates (e.g., manganese bisglycinate chelate, manganese glycinate chelate, manganese aspartate, manganese gluconate, manganese picolinate, manganese sulfate, manganese citrate, or manganese chloride. In an embodiment, the metal is copper and a copper compound may include a cupric oxide, cupric sulfate, copper amino acid chelates, and copper gluconate. In still another embodiment, the metal is iron and iron may exist in various forms such as ferrous and ferric iron salts (for example, ferrous sulfate, ferrous gluconate, ferric citrate, or ferric sulfate cobalt). In an embodiment, the metal is cobalt and a cobalt compound may include cobalt acetate, cobalt sulfate, cobalt picolinate, cobalt orotate, or cobalt citrate. In an embodiment, the metal is chromium and a chromium compound may include chromium chloride, chromium nicotinate, chromium picolinate, high-chromium yeast, or chromium citrate.

In some embodiments, the CHP may be contained together with a probiotic, or may be administered in combination with a probiotic. The may be selected from the group consisting of Lactobacillus rhamnosus, Lactobacillus acillus, Bifidobacterium lactus, Bifidobacterium longum, or other commercially available probiotics.

The term probiotic as used herein refers to live probiotic bacteria, non-replicating probiotic bacteria, dead probiotic bacteria, non-viable probiotic bacteria, fragments of probiotic bacteria such as DNA, metabolites of probiotic bacteria, cytoplasmic compounds of probiotic bacteria, cell wall materials of probiotic bacteria, culture supernatants of probiotic bacteria, and/or combinations of any of the foregoing. The probiotic may for example be live probiotic bacteria, non-replicating probiotic bacteria, dead probiotic bacteria, non-viable probiotic bacteria, or any combination thereof. In an embodiment of the disclosure the probiotic is live probiotic bacteria.

Additional vitamins and minerals may also be administered in combination with CHP. For example, the composition may contain one or more of the following micronutrients, calcium, magnesium, phosphorus, iron, zinc, copper, iodine, selenium, vitamin A or retinol activity equivalent (RAE) for example in the form of β carotene or a mix of carotenoids, Vitamin C, Vitamin B1, niacin, folic acid, biotin, Vitamin E, vitamin B2, vitamin B6, vitamin B15, vitamin D, iron, zinc.

In an embodiment of the disclosure, the CHP is administered in combination with an ingredient selected from the group consisting of vitamin B2, vitamin B6, vitamin B12, vitamin D, magnesium, iron, zinc, arachidonic acid, histidine, arginine, glycine, serine or combinations thereof.

In a further embodiment of the present disclosure, the CHP is administered in the form of a composition. Such composition may comprise any other ingredient for example one or more ingredients set out herein e.g. probiotics vitamins and minerals. The composition may also comprise other ingredients commonly used in the form of composition in which it is employed e.g., a powdered nutritional supplement, a food product, or a dairy product. Non limiting examples of such ingredients include: other nutrients, for instance, selected from the group of lipids (optionally in addition to DHA and ARA), amino acids such as histidine, carbohydrates, and protein, micronutrients (in addition to those set out above), or pharmaceutically active agents; conventional food additives such as anti-oxidants, stabilizers, emulsifiers, acidulants, thickeners, buffers or agents for pH adjustment, chelating agents, colorants, excipients, flavor agents, osmotic agents, pharmaceutically acceptable carriers, preservatives, sugars, sweeteners, texturizers, emulsifiers, water and any combination thereof.

In an embodiment of the disclosure the composition is a product selected from the group consisting of a nutritional product, a food product, a functional food product, a healthy ageing product, a nutritional supplement, a pharmaceutical formulation, a beverage product, and a pet food product.

The term “nutritional product”, as used herein, means any product that can be used to provide nutrition to a subject. Typically, nutritional products contain a protein source, a carbohydrate source and a lipid source.

The term “food product”, as used herein, refers to any kind of product that may be safely consumed by a human or an animal. Said food product may be in solid, semi-solid or liquid form and may comprise one or more nutrients, foods or nutritional supplements. For instance, the food product may additionally comprise the following nutrients and micronutrients: a source of proteins, a source of lipids, a source of carbohydrates, vitamins and minerals. The composition may also contain anti-oxidants, stabilizers (when provided in solid form) or emulsifiers (when provided in liquid form).

The term “functional food product”, as used herein, refers to a food product providing an additional health-promoting or disease-preventing function to the individual.

The term “healthy ageing product”, as used herein, refers to a product providing an additional health-promoting or disease-preventing function related to healthy ageing to the individual.

The term “pharmaceutical formulation” as used herein, refers to a composition comprising at least one pharmaceutically active agent, chemical substance or drug. The pharmaceutical formulation may be in solid or liquid form and can comprise at least one additional active agent, carrier, vehicle, excipient, or auxiliary agent identifiable by a person skilled in the art. The pharmaceutical formulation can be in the form of a tablet, capsule, granules, powder, liquid, spray, aerosol, or syrup.

The term “beverage product” as used herein, refers to a nutritional product in liquid or semi-liquid form that may be safely consumed by an individual.

The term “pet food product” as used herein refers to a nutritional product that is intended for consumption by pets. A pet, or companion animal, as referenced herein, is to be understood as an animal selected from dogs, cats, birds, fish, rodents such as mice, rats.

The term “nutritional supplement” as used herein, refers to a nutritional product that provides nutrients to an individual that may otherwise not be consumed in sufficient quantities by said individual. For instance, a nutritional supplement may include vitamins, minerals, fiber, fatty acids, or amino acids. Nutritional supplements may for example be provided in the form of a pill, a tablet, a lozenge, a chewy capsule or tablet, a tablet or capsule, or a powder supplement that can for example be dissolved in water or sprinkled on food. Nutritional supplements typically provide selected nutrients while not representing a significant portion of the overall nutritional needs of a subject. Typically, they do not represent more than 0.1%, 1%, 5%, 10% or 20% of the daily energy need of a subject. A nutritional supplement may be used during pregnancy, e.g., as a maternal supplement.

In exemplary embodiments, the pharmaceutical composition of the embodiments can be administered in a variety of ways, including orally, topically, parenterally, intravenously, intradermally, colonically, rectally, intramuscularly, transdermally, intranasal rout, or intraperitoneally.

The pharmaceutical composition may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The formulation may take such forms as suspensions, solutions or emulsions in oily or aqueous vehicles, and may contain agents such as suspending, stabilizing and/or dispersing agents.

For example, a parenteral preparation may be a sterile injectable solution or suspension in a nontoxic parenterally acceptable diluent or solvent. Among the acceptable vehicles and solvents that may be employed are water, 0.9% saline solution, or other suitable aqueous media.

In one embodiment, the concentration of the intravenous solution formulation is from about 1 mg/liter to about 200 mg/ml, from about 5 mg/ml to about 150 mg/ml, from about 10 mg/ml to about 100 mg/ml. In another embodiment, the concentration of the intravenous solution formulation is about 1 mg/liter, about 2 mg/liter, about 3 mg/liter, about 4 mg/liter, about 5 mg/liter, about 6 mg/liter, about 7 mg/liter, about 8 mg/liter, about 9 mg/liter, about 10 mg/liter, about 11 mg/liter, about 12 mg/liter, about 13 mg/liter, about 14 mg/liter, about 15 mg/liter, about 20 mg/liter, about 25 mg/liter, about 30 mg/liter, about 35 mg/liter, about 40 mg/liter, about 45 mg/liter, about 50 mg/liter, about 55 mg/liter, about 60 mg/liter, about 65 mg/liter, about 70 mg/liter, about 75 mg/liter, about 80 mg/liter, about 85 mg/liter, about 90 mg/liter, about 95 mg/liter, about 100 mg/liter, about 110 mg/liter, about 120 mg/liter, about 130 mg/liter, about 140 mg/liter, about 150 mg/liter, about 160 mg/liter, about 170 mg/liter about 180 mg/liter, about 190 mg/liter, or about 200 mg/liter.

In another embodiment, the pharmaceutical composition may be formulated into a diffusion (slow drip) formulation or an intravenous bolus injection.

In yet another embodiment, the pharmaceutical composition may be administered orally or formulated for oral administration. Administration may be via immediate release tablets and capsule or enteric coated tablets or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, sterile injectable solutions and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, hydroxypropyl cellulose, polyvinylpyrrolidone, cellulose, USP or sterile water, syrup base and methyl cellulose. The formulations can additionally include: lubricating agents such as talc, magnesium stearate, and stearic acid; wetting agents; emulsifying and suspending agents; preserving agents such as methyl and propylhydroxy-benzoates; sweetening agents; and flavoring agents.

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject.

In some embodiments, the pharmaceutical compositions are formulated in a unit dosage form. The term “unit dosage forms” refers to physically discrete units suitable as unitary dosages for human subjects and other mammals, each unit containing a predetermined quantity of active material (therapeutically effective amount) calculated to produce the desired therapeutic effect, in association with a suitable pharmaceutical excipient (e.g., a tablet, capsule, ampoule). The compounds are generally administered in a pharmaceutically effective amount. In some embodiments, each dosage unit contains from about 1 mg to about 100 mg of a CHP compound. In some embodiments, each dosage unit contains from about 2 mg to about 60 mg, from about 3 mg to about 50 mg, from about 4 mg to about 40 mg, from about 5 mg to about 30 mg, from about 6 mg to about 20 mg, from about 8 mg to about 15 mg, or from about 8 mg to about 10 mg of a CHP compound.

In other embodiments, each dosage unit contains about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 30 mg, about 40 mg, about 50 mg, about 60 mg, about 70 mg, about 80 mg, about 90 mg, about 100 mg, about 200 mg, about 300 mg, about 400 mg, about 500 mg, about 700 mg, about 800 mg, about 900 mg, about 1000 mg, about 1500 mg, about 2000 mg, or about 3000 mg of a CHP compound.

In embodiments, with regard to any of the above-discussed methods, compositions, and uses, CHP may be administered to the subject in an amount from about 0.001 to about 3000 mg/kg. In some embodiments, the effective amount of the CHP may be about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg. The amounts are based on the amount of anhydrous CHP.

In embodiments, with regard to any of the above-discussed methods, composition, and uses, an effective amount of zinc ranges from about 0.1-1 mg/day, about 1-10 mg/day, 10-50 mg/day, 50-100 mg/day, 100-150 mg/day, 150-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, or 1900-2000 mg/day, as calculated in term of zinc cation.

In some embodiments, for a composition comprising CHP and zinc or for an administration of a CHP-containing composition and a Zn-containing composition, the weight ratio of zinc to CHP is from about 1:10 to about 100:1 (as calculated in terms of anhydrous CHP and zinc element, unless otherwise indicated). In some embodiments, the weight ratio of zinc to CHP is from about 1:6 to about 5:1. In some embodiments, the weight ratio of zinc to CHP is from about 1:15 to about 20:1. In some embodiments, the weight ratio of zinc to CHP is from about 1:30 to about 4:1. In some embodiments, the weight ratio of zinc to CHP is from about 1:8 to about 4:1. In some embodiments, the weight ratio of zinc to CHP is from about 1:40 to about 40:1. Zinc as noted above relates to the amount of zinc cation.

For preparing solid compositions such as tablets, the active principle ingredient is mixed with a pharmaceutical excipient to form a solid mixed-blend composition containing a homogeneous mixture of a compound of the present disclosure. When referring to these mixed-blend compositions as homogeneous, it is meant that the active ingredient is dispersed evenly throughout the composition so that the composition may be readily subdivided into equally effective unit dosage forms such as tablets, pills and capsules.

The tablets or pills of the present disclosure may be powder-coated or otherwise compounded to provide a dosage form affording the advantage of prolonged action or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. In one embodiment, the film coating is a polyvinyl alcohol-based coating.

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

Suitable excipients include binders, fillers, disintegrants, lubricants, antioxidants, chelating agents, and color agents.

Further advantages and features of the present invention are apparent from the figures and non-limiting examples.

### EXAMPLES

### Example 1: Establishment of β-Cell Dysfunction Mouse Model and Administration of Cyclo-Z

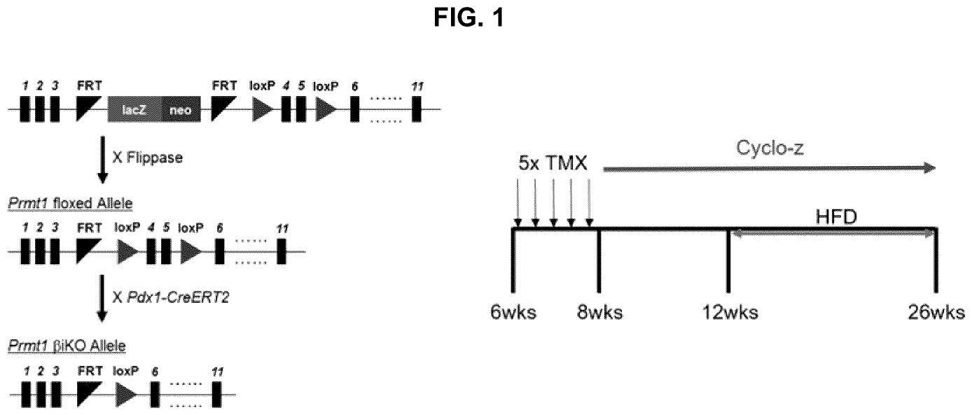

Prmt1-flox mice capable of deleting the Prmt1 gene were crossed with Pdx1-CreERT2 mice to construct Prmt1 BiKO (Prmt1-flox; Pdx1-CreERT2) mice, and treated with tamoxifen to delete specificity Prmt1 gene in β cells of adult mice. (FIG. 1). The high fat diet (HFD) was administered for 14 weeks from 12 to 26 weeks of age, and the development process of β-cell failure was observed through metabolic phenotype and histological analysis of β cells. It was considered that the β-cell failure started from the time Prmt1 was removed from the B cells, and the phenotype was observed while administering the drug to the comparative group (vehicle administration group) and a Cyclo-Z (a combination of CHP and zinc) administration group from 8 weeks of age (FIG. 1).

### Example 2: Blood Glucose Improvement Effect by Cyclo-Z

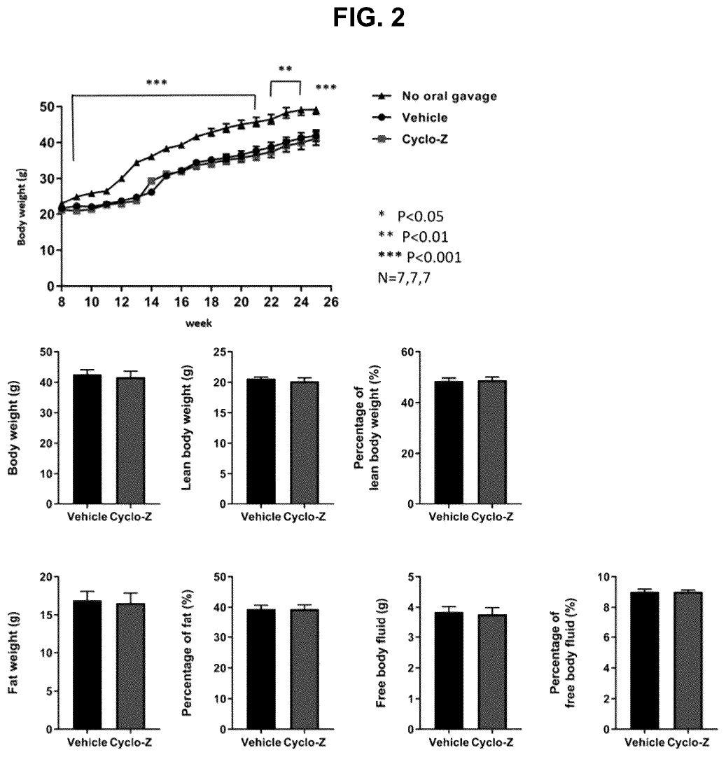

There was no difference in body weight between the comparative group and the Cyclo-Z administered group during the period of 8 to 14 weeks of high-fat diet intake, and there was no difference between the two groups in the results of body composition analysis at 26 weeks of age (FIG. 2).

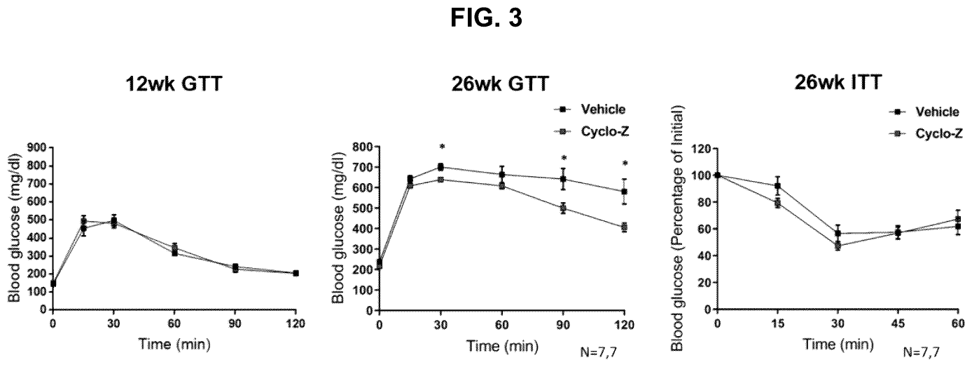

As a result of the glucose tolerance test (GTT), there was no difference in glucose tolerance between the comparative group and the Cyclo-Z administration group at 12 weeks of age, the initial stage of Cyclo-Z administration. (FIG. 3). As a result of the insulin tolerance test (ITT), there was no difference in insulin resistance between the two groups, indicating that the blood sugar improvement effect of Cyclo-Z was due to the direct protective effect on β cells rather than weight control or insulin sensitivity improvement.

### Example 3: Inhibition of β-Cell Dysfunction by Cyclo-Z or Ameliorating β-Cell Failure

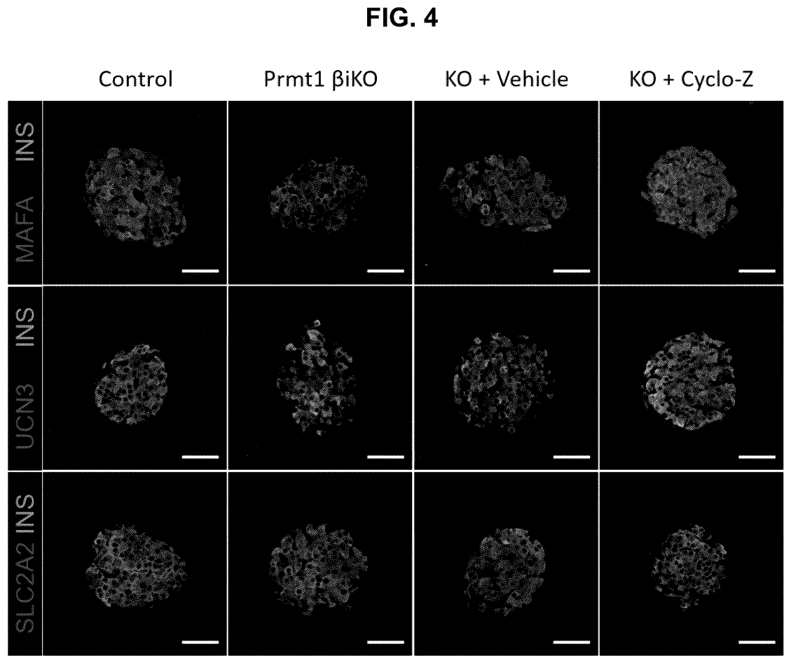

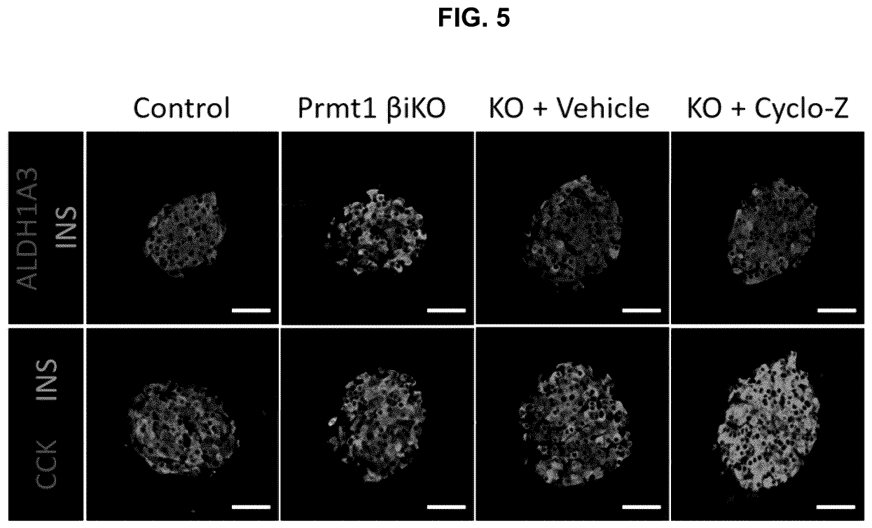

β-cell failure is accompanied by loss of identity and dematuration of β-cells. Loss of identity of β-cells can be detected by reduced expression of genes that play an important role in β-cell function and abnormal expression of genes indicating dematuration. The inventors have observed using immunofluorescence staining that the expression of MAFA, UCN3, and SLC2A2 proteins, each related to β-cell function, were reduced in the pancreas of 26-week-old control mice (KO+Vehicle group), but the reduced expressions were suppressed in the CycloZ-administered group. In addition, the expression of ALDHIA3 and CCK proteins, which are indicative of dematuration of β-cells, were significantly reduced in the Cyclo-Z-administered group compared to the vehicle administration group (FIGS. 4 and 5). These results demonstrate that the administration of Cyclo-Z has the effect of suppressing β-cell failure.

### Example 4: Stress Reduction Effect in β-Cells by Cyclo-Z Administration

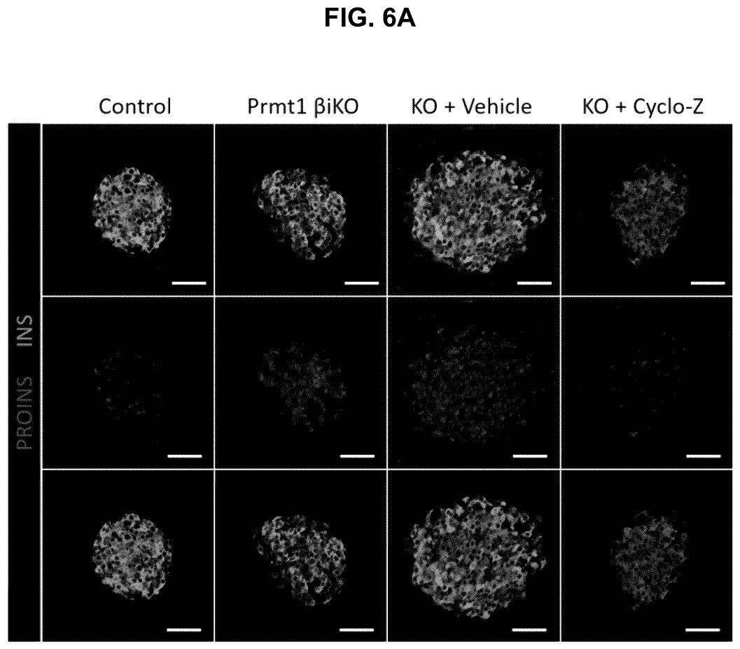

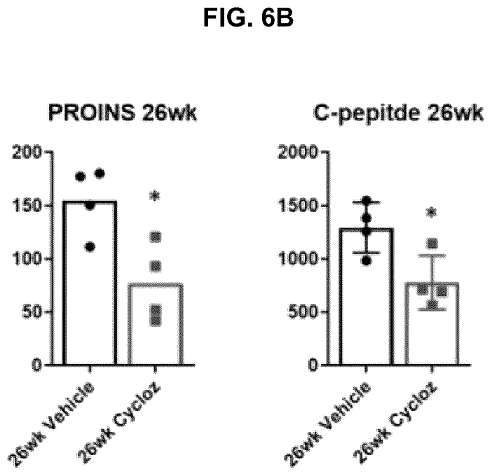

During the progression of type 2 diabetes, proinsulin, a precursor of insulin, is abnormally increased due to decreased function of β-cells, which is also observed in the PRMT1 BiKO mouse model fed a high-fat diet. In 26-week-old control mice, high levels of proinsulin were present in plasma and B cells, but significantly decreased in the Cyclo-Z-administered group (FIGS. 6A and 6B).

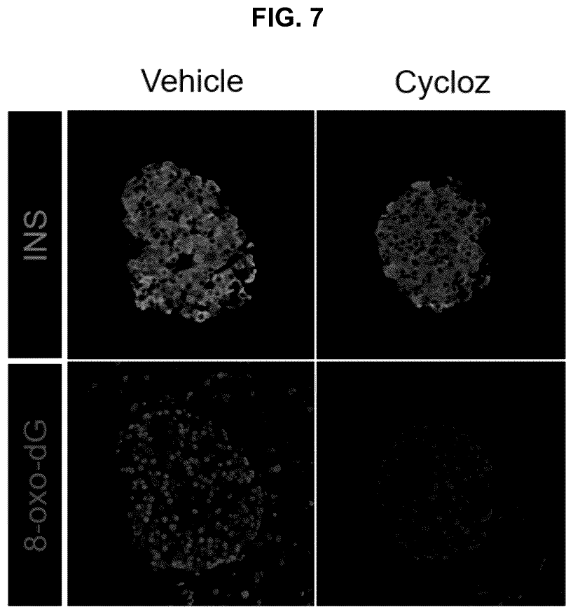

Abnormal increase in proinsulin in β-cells is known to induce intracellular stress. Immunofluorescent staining of 8-oxo-dG (8-Oxo-7,8-dihydro-2′-deoxyguanosine) was performed to confirm the increase in oxidative stress in β-cells due to the increase in proinsulin, and the effects of CHP in reducing oxidative stress in the Cyclo-Z-administered group which showed great decreases in the degree of oxidative stress (FIG. 7). From these results, it can be seen that administration of Cyclo-Z can protect β-cells by suppressing the increase in proinsulin caused by the decline of β-cells function and reducing oxidative stress applied to β-cells.

### Example 5: Beta-Cell Transcriptome Analysis Showing Protective Effects of Cyclo-Z

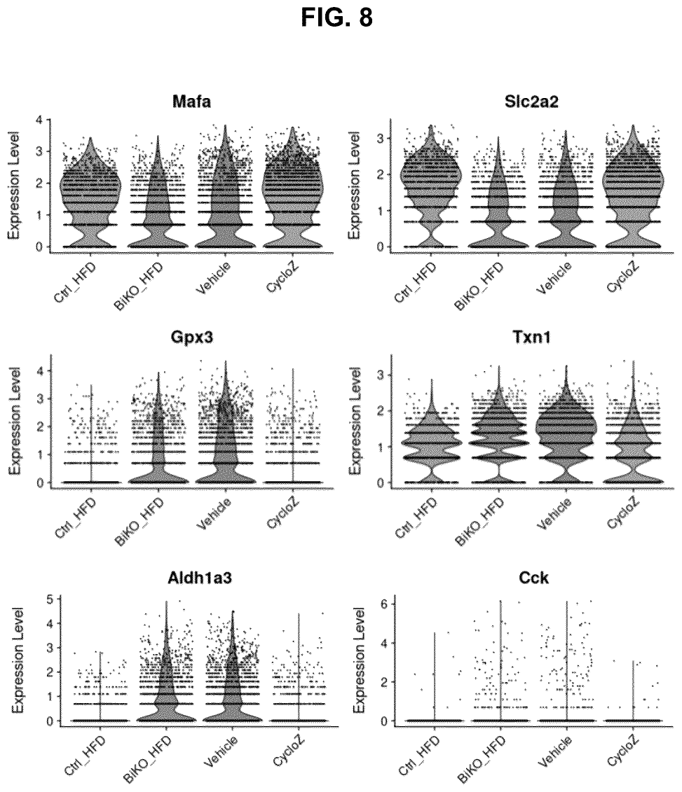

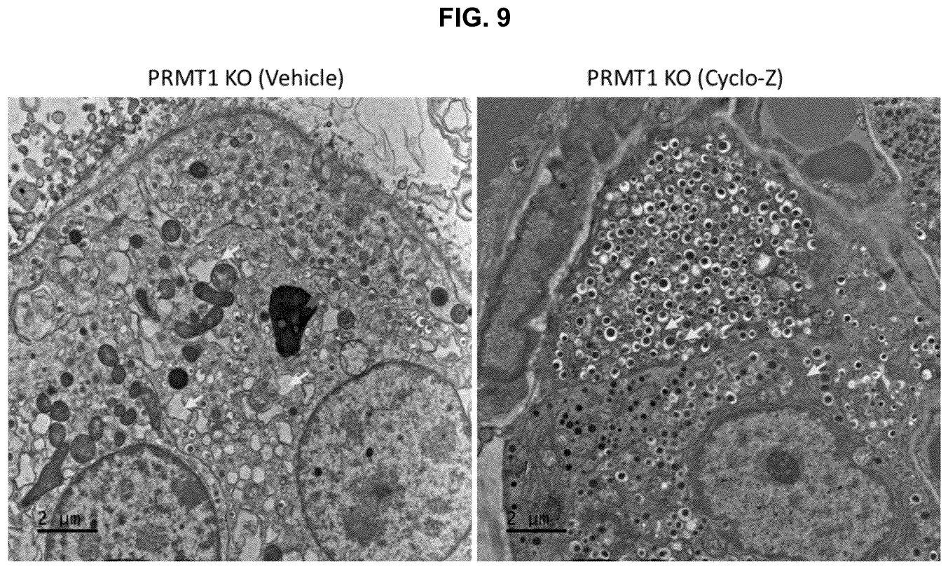

The effect of Cyclo-Z was confirmed at the transcriptome level using single-cell RNA-sequencing analysis using pancreas. Compared to the vehicle group, the expression of Mafa and Slc2a2, which are genes related to β-cell identity, were maintained at high levels in the Cyclo-Z-administered group, while the expression of Aldh1a3 and Cck, which indicate β-cell dematuration, was suppressed in the Cyclo-Z administration group. In addition, the expression of genes belonging to the Gpx (GSH peroxidase) and Txn (Thioredoxin) gene groups, which are known to be expressed to protect cells when reactive oxygen species are generated, was increased in the Cyclo-Z administration group, indicating the reduction of oxidative stress in β-cells (FIG. 8).

### Example 6: Transmission Electron Microscopy (TES) Analysis of Cyclo-Z Protective Effects

The protective effect of β-cell organelles by Cyclo-Z was confirmed through transmission electron microscopy (TES). Compared to the vehicle group, the density of insulin granules increased in the group administered with Cyclo-Z, and dense core granules and halo structures, which are characteristics of mature insulin granules, were observed. Through this, it was confirmed that administration of Cyclo-Z directly acts on β-cells to stabilize and mature insulin granules. In addition, the present inventors surprisingly found through the TES images that Cyclo-Z administration improves the endoplasmic reticulum expansion and destruction, the mitochondrial cristae structure increase, the accumulation of autophagic vesicles in β-cells, all of which were typical phenomenon observed as β-cell failure occurs.

### Example 7: Prmt1 Deletion Induces Changes in Beta Cell Identity

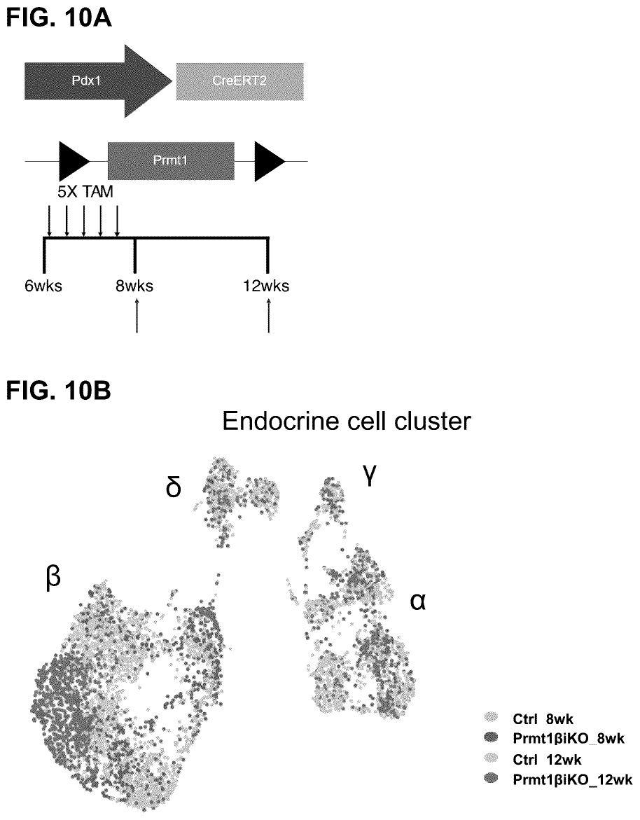

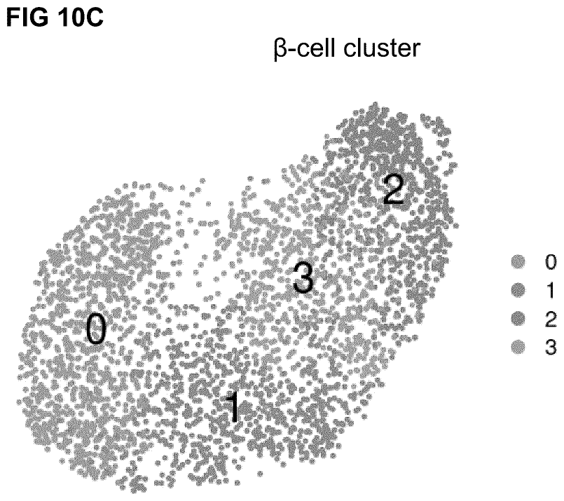

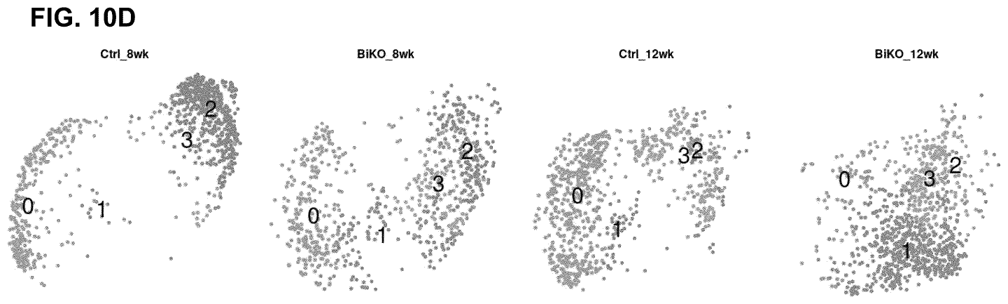

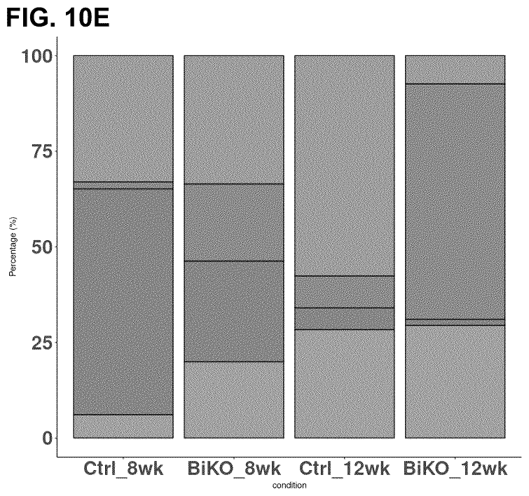

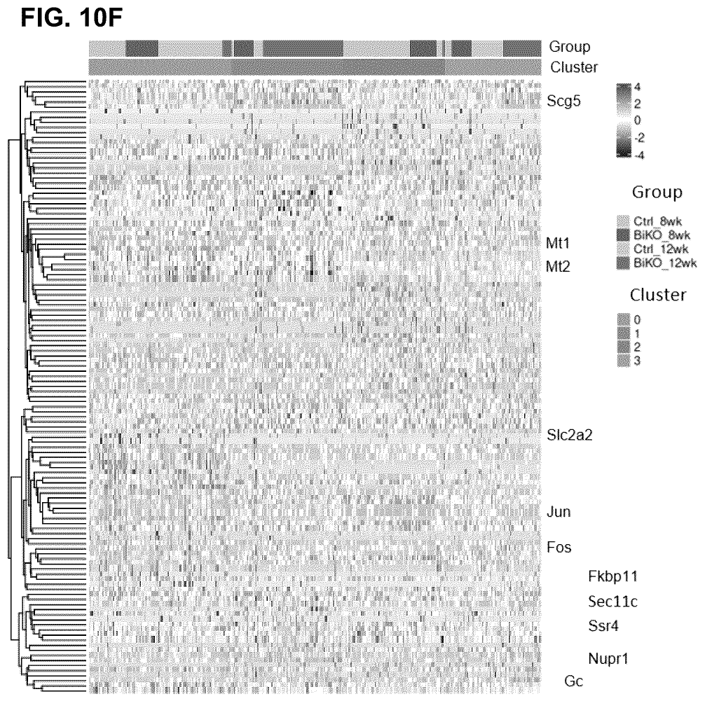

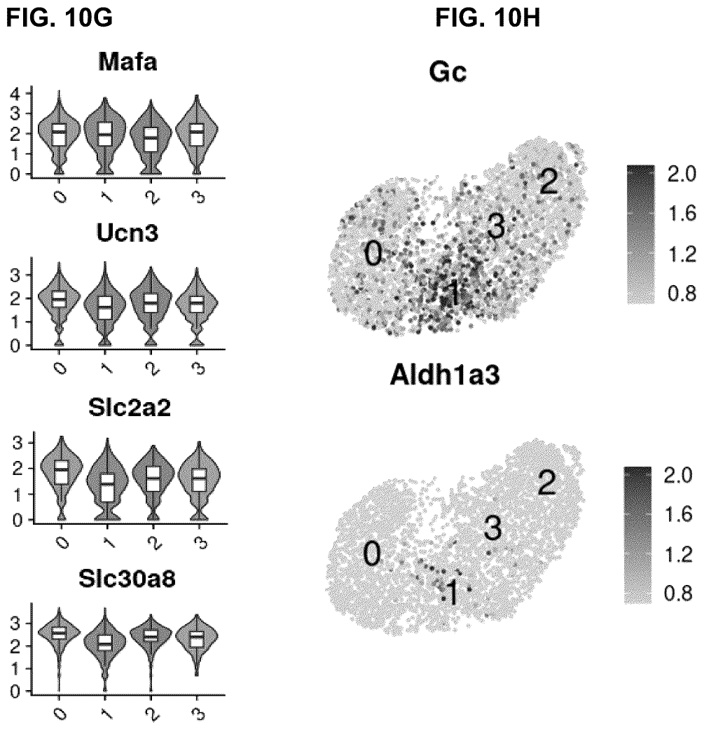

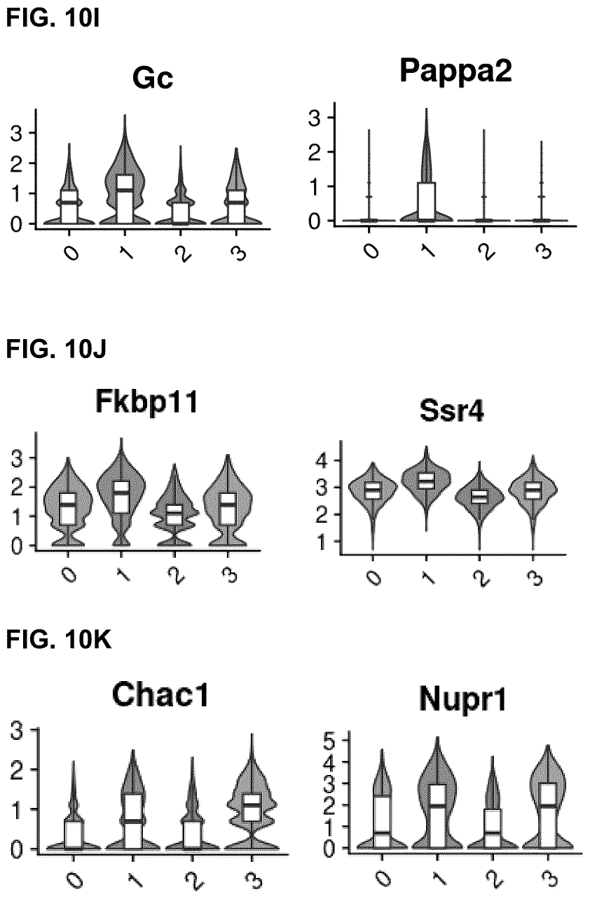

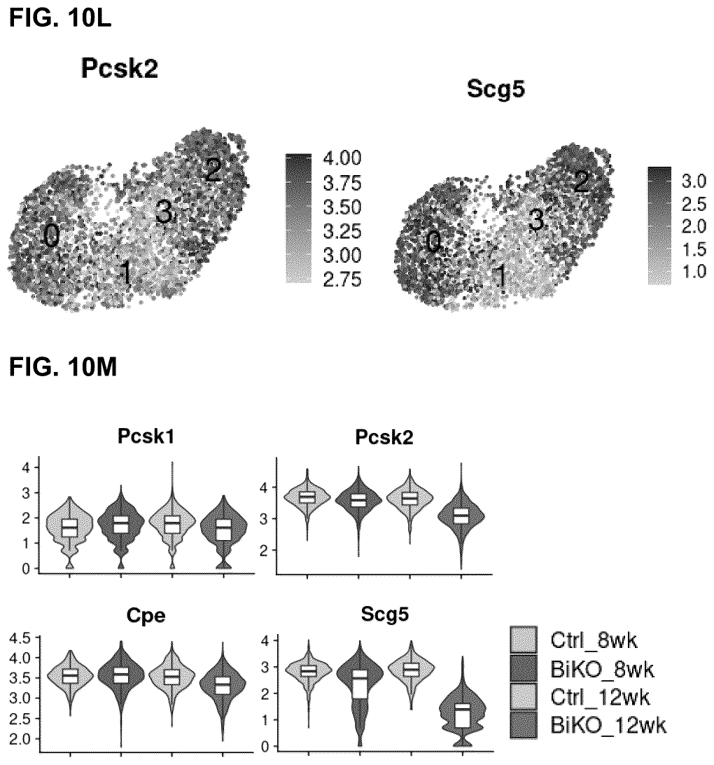

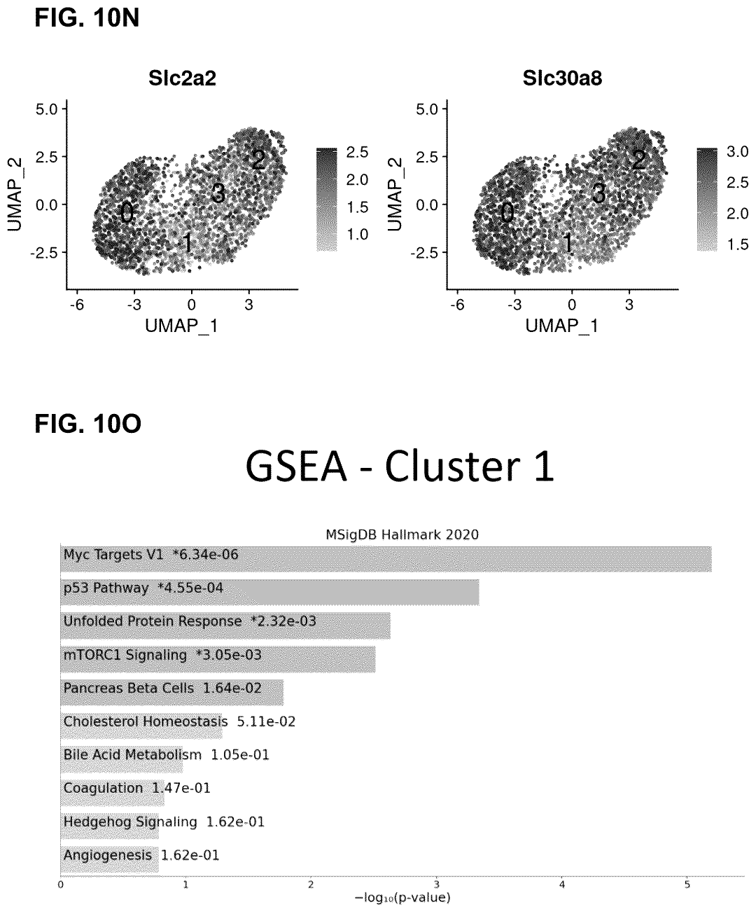

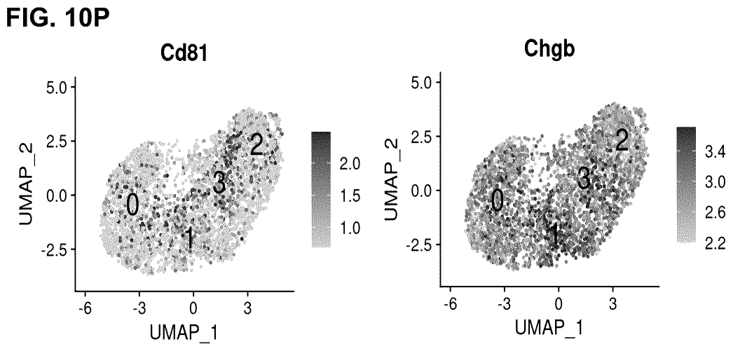

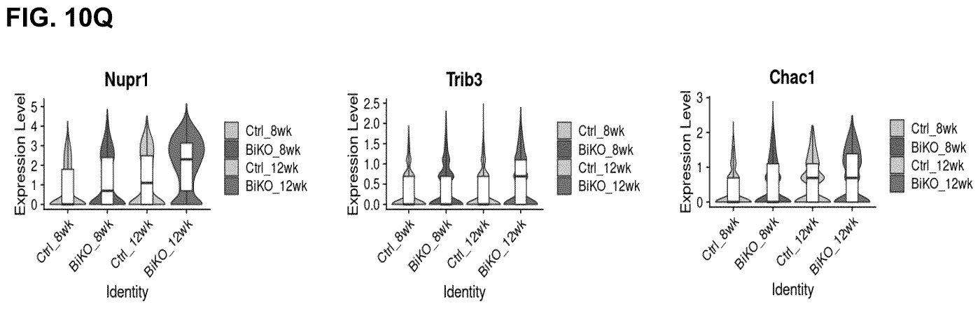

To elucidate molecular mechanisms underlying loss of beta cell identity induced by Prmt1 deletion (Diabetes, 2020), we used inducible b-cell-specific Prmt1 knockout mouse model generated by crossing Prmt1fl/fl mice with Pdx1-CreERT2 mice (Prmt1BiKO) and performed single cell RNA-sequencing (scRNA-seq) analyses using islets isolated from Control and Prmt1BiKO mice at 8 and 12 weeks of age to analyze effects of Prmt1 deletion on beta cell (FIG. 10A). Uniform Manifold Approximation and Projection (UMAP) plotting of endocrine cells exhibited that transcriptional differences between Control and Prmt1BiKO mice are prominent in beta cells and less obvious in other endocrine cell types (FIG. 10B). After isolating beta cells, we identified four clusters by unsupervised clustering (FIG. 10C). Among the beta cell clusters, cluster 1 is mostly derived from Prmt1BiKO mice at 12 weeks of age (FIGS. 10D and 10E). Gene expression analysis showed down-regulation of genes involved in insulin secretion (Slc2a2, Slc30a8) in cluster 1 (FIGS. 10F, 10G, and 10N). GSEA analysis of cluster 1 revealed up-regulation of Myc target genes including ribosomal subunits (Rpl, Rps) (FIG. 10O).

Based on the recent finding that transgenic mouse over-expressing Myc gene in beta cell showed signs of beta cell immaturity (Matthias Hebrok, Nat Comm 2018), it can be speculated that loss of maturity was already provoked in beta cells of Prmt1BiKO mice around 12 weeks of age. Indeed, we identified aberrant expression of alpha-cell marker genes (Gc, Ttr, and Pappa2) and immature beta cell marker genes (Gc and Aldh1a3) in cluster 1 (FIGS. 10H, 10K, and 10P). Moreover, genes involved in unfolded protein response (UPR) and downstream signaling of IRE1/XBP1 pathway (Fkbp11 and Ssr4) or PERK/ATF4 pathways (Trib3, Chac1) were up-regulated in cluster 1 (FIGS. 10J, 10K, and 10Q). Notably, expression of Nupr1, a stress-sensitive protein involved in ATF4 and p53 pathway, was significantly increased in Prmt1-deficient beta cells (FIG. 10Q) (Päth G et al., NUPR1 preserves insulin secretion of pancreatic β-cells during inflammatory stress by multiple low-dose streptozotocin and high-fat dict. Am J Physiol Endocrinol Metab. 2020 Aug. 1; 319(2):E338-E344). These results indicate that loss of Prmt1 causes ER stress and provokes changes in mature identity of beta cells without HFD-diet induced metabolic stress.

### Example 8: Proinsulin Processing was Impaired in Prmt1BiKO Islets

Interestingly, Prmt1 deletion induced down-regulation of genes involved in insulin processing, such as Pcsk2 and Scg5 (FIGS. 10L and 10M). Proinsulin is processed into mature insulin and c-peptide by combined action of prohormone convertases (PC)1/3, PC2, and carboxypeptidase E (CPE). Impairment of these process is considered as a key feature of beta cell dysfunction that can be observed in early stage of type 2 diabetes (SERCA-Molina-Diabetologia 2023, CPE-Verchere-Diabetes 2023). PC2 is encoded by Pcsk2 gene and its activation requires chaperone protein 7B2 which is encoded by Scg5 gene. Scg5-null mouse showed impaired activation of PC2 and elevated level of des-31,32 proinsulin intermediate (Westphal C H, et al., The neuroendocrine protein 7B2 is required for peptide hormone processing in vivo and provides a novel mechanism for pituitary Cushing's disease. Cell. 1999 Mar. 5:96(5):689-700).

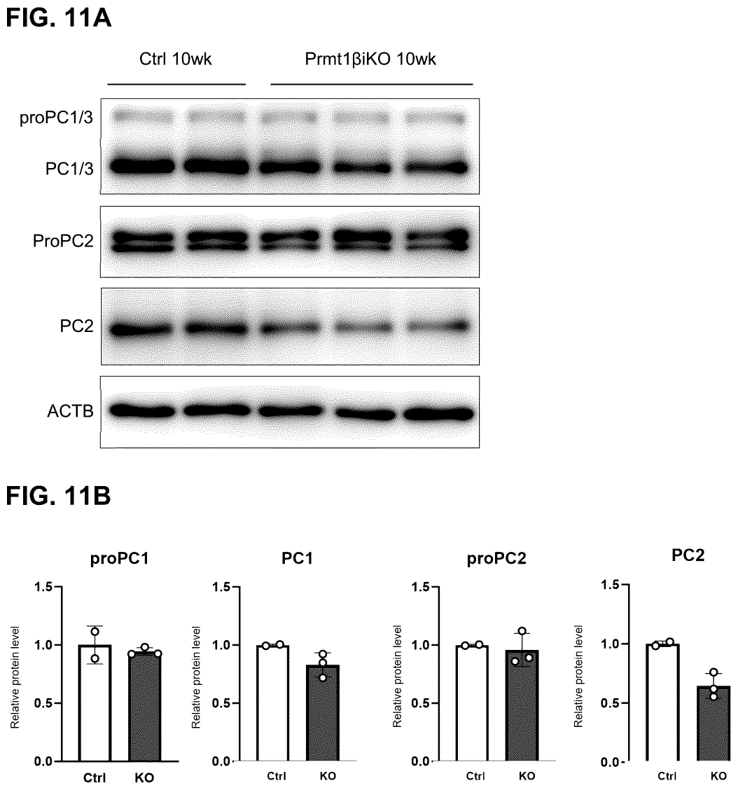

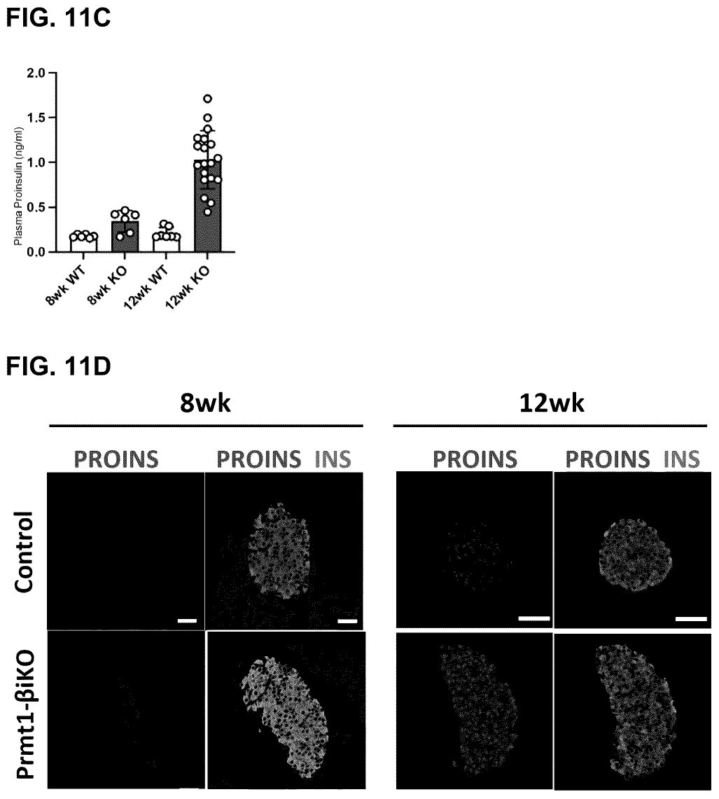

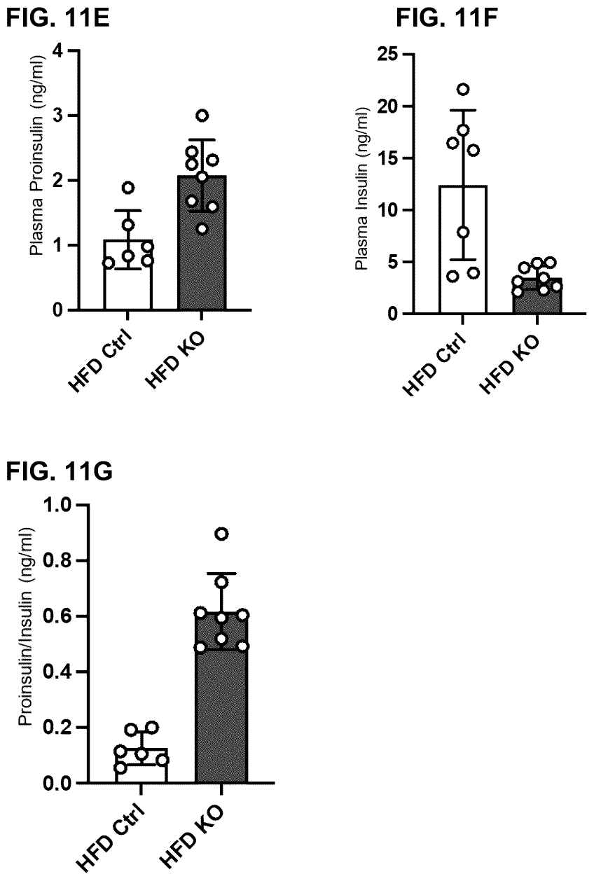

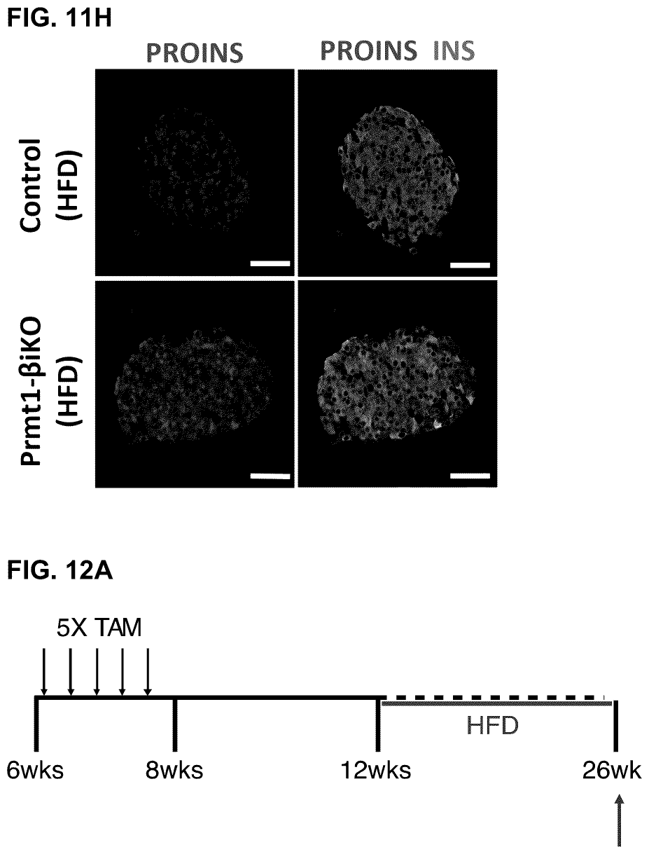

To analyze the effects of Scg5 down-regulation in Prmt1BiKO mice, we isolated islets from 10 wk-weeks-old mice produced in Example 1 or Example 7 and analyzed protein levels of proinsulin processing enzymes.

Whereas premature form of PC2 protein level is comparable between Control and Prmt1BiKO islets, active PC2 protein level was decreased in Prmt1BiKO islets. ProPC1/3 and PC1/3 protein levels were analyzed to determine presence of post-translational regulatory mechanisms of prohormone convertases, but their protein levels were not changed (FIGS. 11A and 11B). In line with these results, plasma proinsulin level was increased in Prmt1 BiKO mice at 8 weeks of age compared to control mice, which was further heightened at 12 weeks of age (FIG. 11C). Immunofluorescence staining exhibited increased proinsulin protein in islets of Prmt1BiKO mice at 12 weeks of age (FIG. 11D).

These results are consistent with previous findings from electron microscopic analysis showing that immature secretory granule was increased in beta cells of Prmt1BiKO mice at 12 weeks of age (Kim H, et al., PRMT1 Is Required for the Maintenance of Mature B-Cell Identity. Diabetes. 2020 March; 69(3):355-368). As chronic HFD feeding aggravated beta cell dysfunction in Prmt1BiKO mice (Kim H, et al., Diabetes 2020), elevation of proinsulin level in Prmt1BiKO mice was also exacerbated by HFD feeding (FIG. 11E). Electron microscopic analysis revealed significant reduction of mature insulin granules in Prmt1BiKO beta cells (FIG. 11I).

Of note, immunofluorescence staining of proinsulin in HFD-fed Prmt1BiKO islets showed increased number of beta cells exhibiting cytoplasmic pattern (ER retention) of intracellular proinsulin compared to control islets where golgi (juxtanuclear) pattern was predominant, which was suggested as a sign of increased misfolded proinsulin (FIG. 11H) (Arunagiri A. et al., Proinsulin misfolding is an early event in the progression to type 2 diabetes. Elife. 2019 Jun. 11; 8:e44532). Plasma insulin level was significantly decreased, and thus proinsulin-to-insulin ratio was markedly increased in HFD-fed Prmt1BiKO mice, suggesting severe impairment of insulin secretion (FIGS. 11F and 11G).

### Example 9: High Fat Diet (HFD) Exacerbates Beta Cell Dedifferentiation in Prmt1BiKO

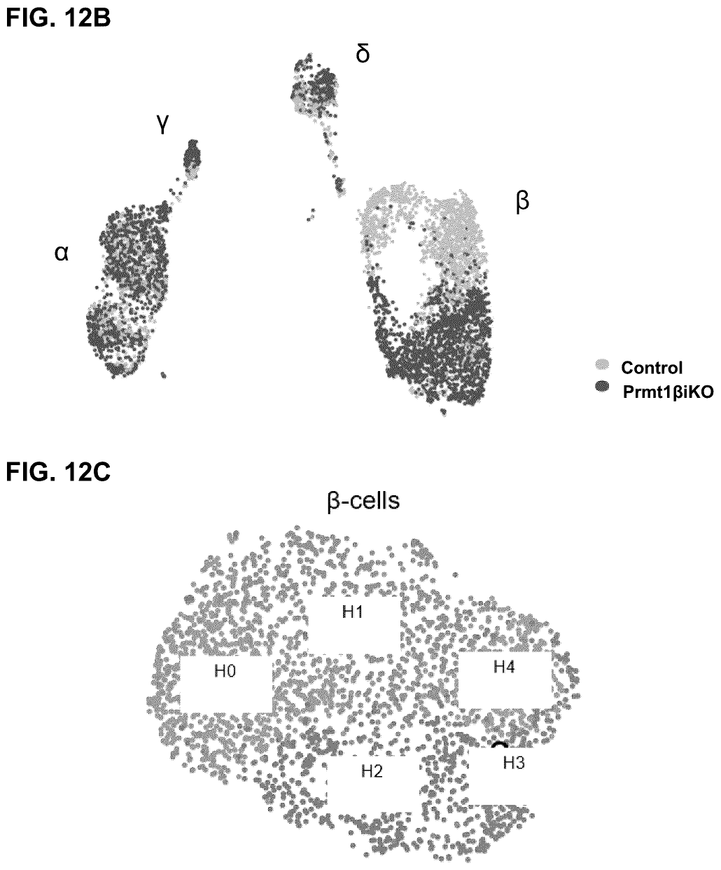

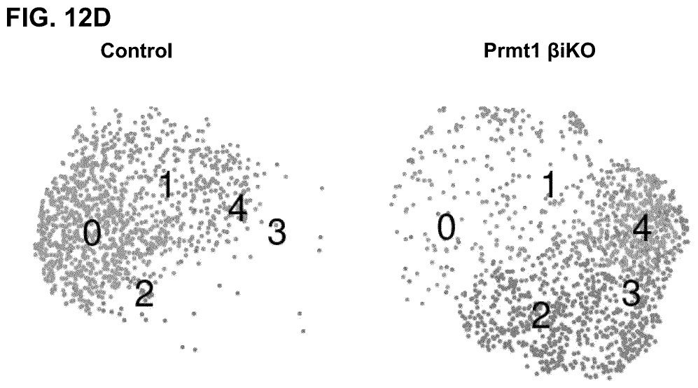

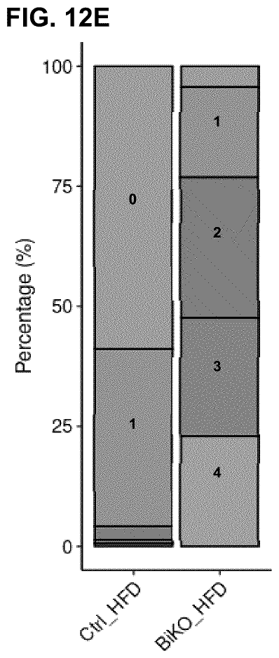

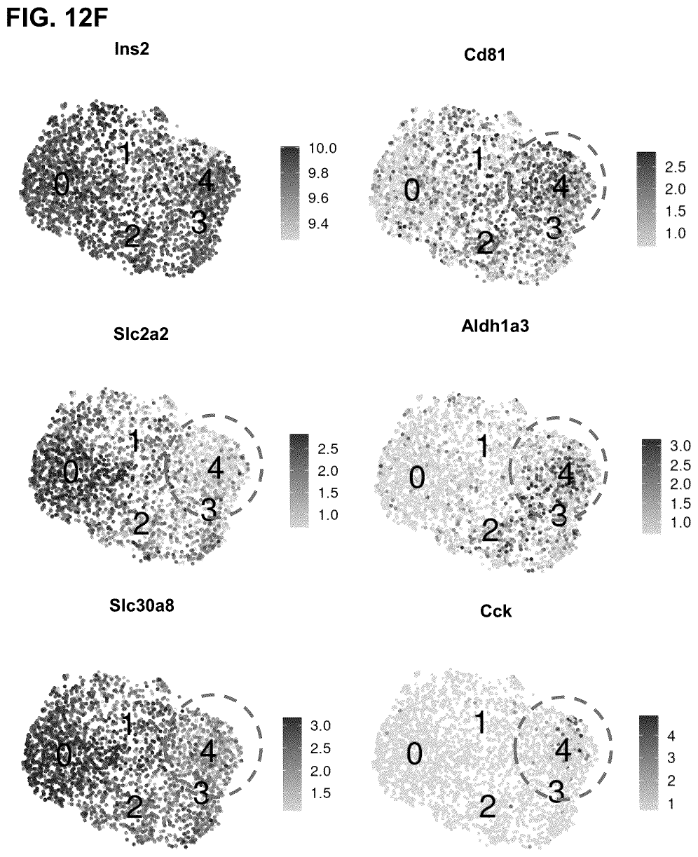

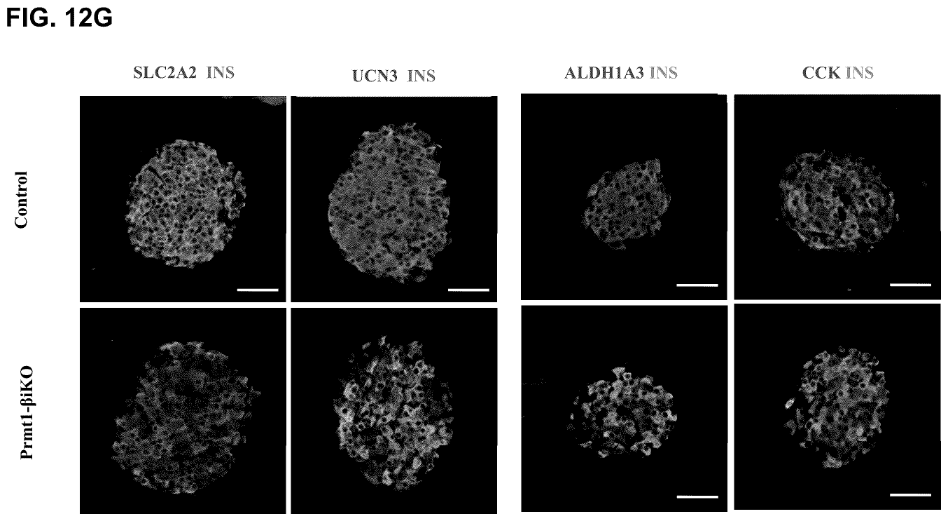

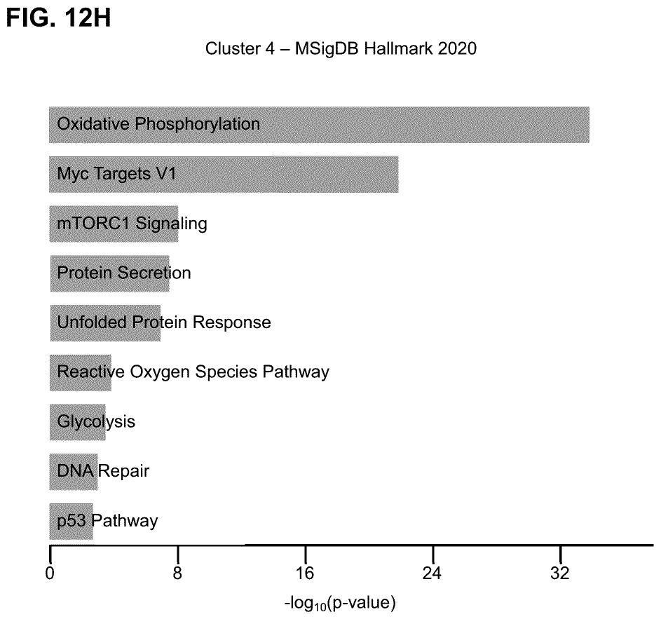

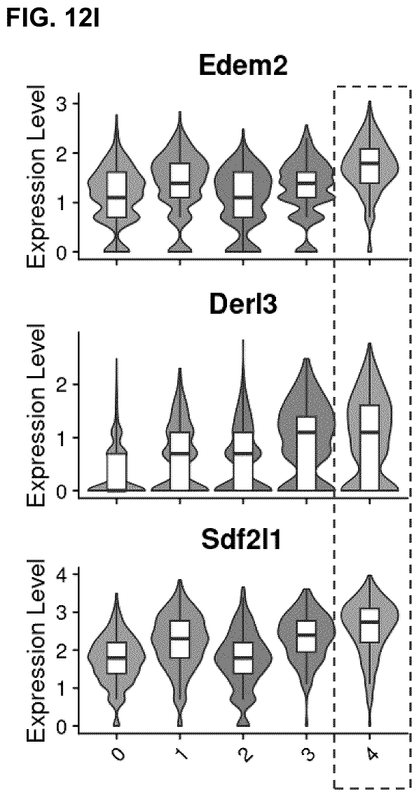

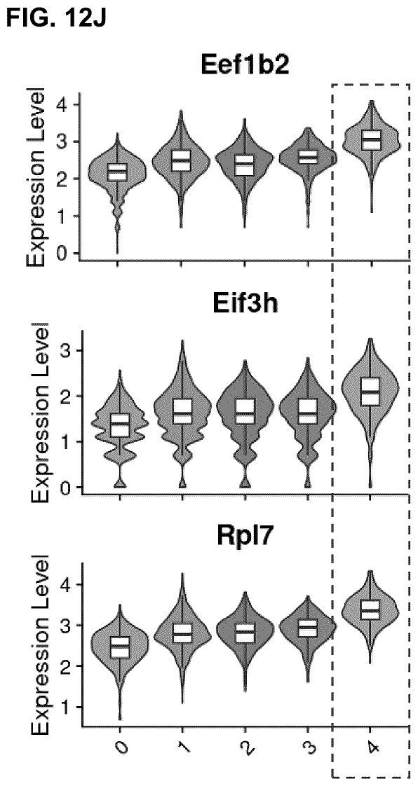

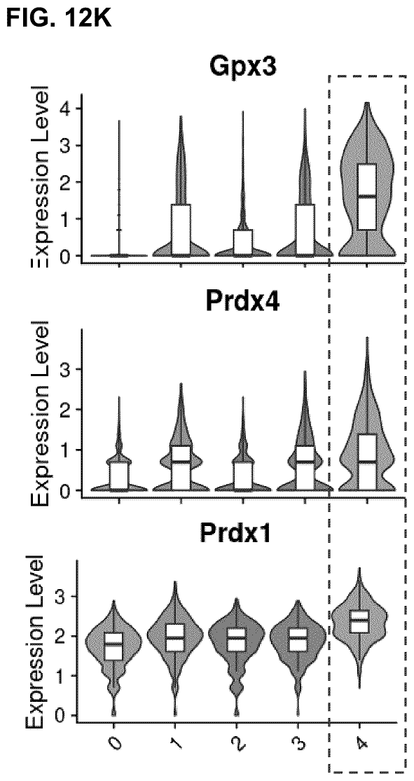

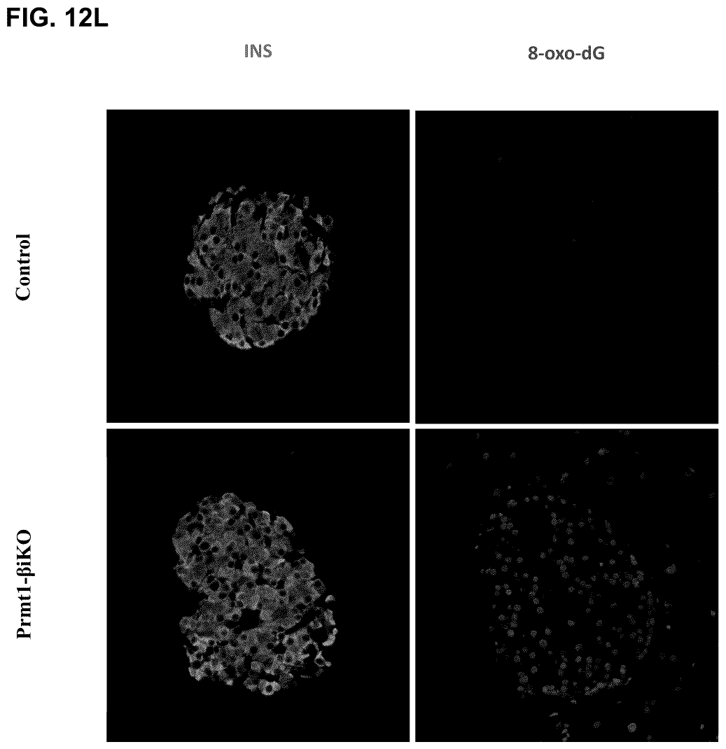

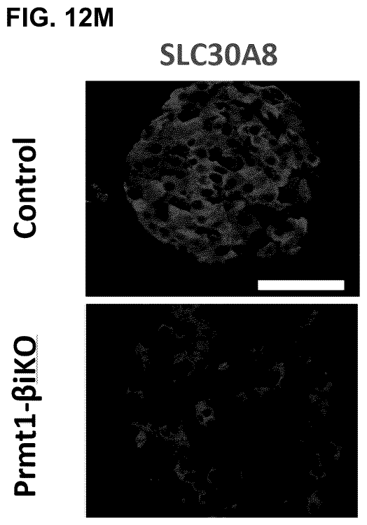

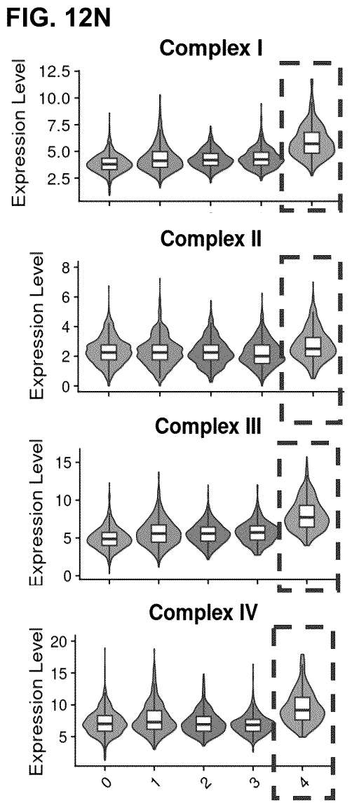

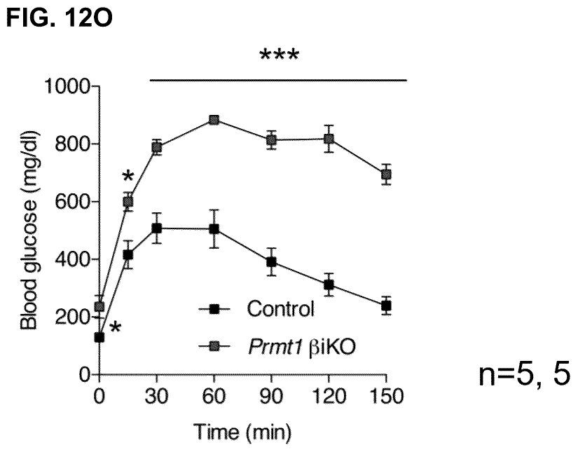

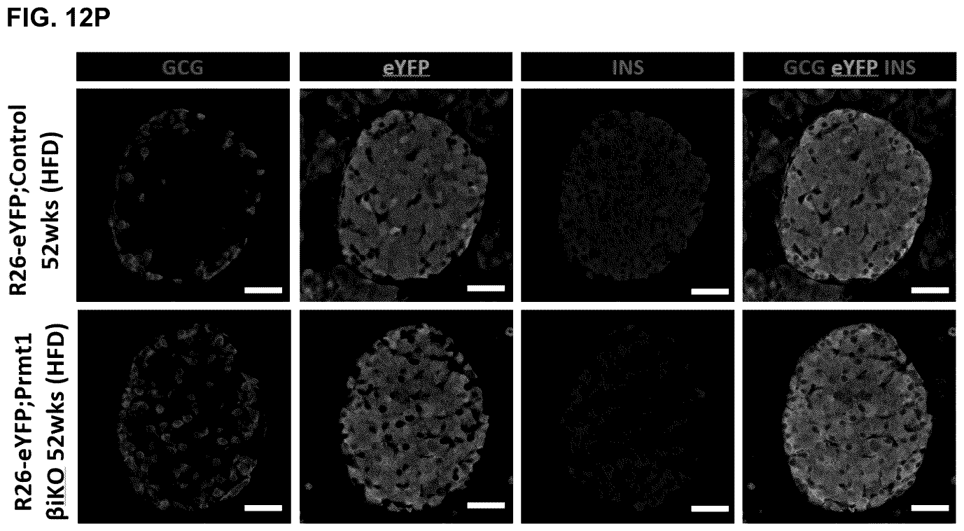

We observed loss of beta cell identity in Prmt1 BiKO mice after long-term HFD feeding (Kim H, et al., Diabetes 2020). To elucidate transcriptomic features of dedifferentiating beta cell, we performed scRNA-seq of islets from HFD-fed Prmt1 BiKO mice (FIG. 12A).

UMAP plot of endocrine cells indicated that endocrine cells other than beta cell showed slight changes in Prmt1BiKO islets compared to control islets (FIG. 12B). After isolating beta cell clusters, we identified five beta cell clusters by unbiased clustering (FIG. 12C). Beta cell clusters of Prmt1BIKO mice was distinguished from those of control mice (FIG. 12D). In control mice, most of beta cells belonged to cluster H0 and H1, but in Prmt1 BiKO mice, these clusters were replaced by cluster H2, H3, and H4 (FIG. 12E).

Gene expression analysis revealed that Cluster H4 exhibited highest degree of beta cell dedifferentiation among the beta cell clusters of Prmt1BiKO mice. Beta cells in cluster H4 showed significant down-regulation of maturity genes (Mafa, Slc2a2, and Slc30a8) and proportion of cells expressing dedifferentiation markers (Cd81, Aldh1a3, and Cck) were markedly increased (FIG. 12F). Immunofluorescence staining of islets revealed decrease of SLC2A2, UCN3, and SLC30a8 protein levels and appearance of beta cells expressing ALDH1A3 and CCK in Prmt1BiKO islets (FIGS. 12G and 12M).

To unveil transcriptomic profile of dedifferentiating beta cell during progressive beta cell dysfunction, GSEA analysis of cluster H4 was performed. The GSEA analysis of cluster H4 revealed up-regulation of Myc targets such as ribosomal proteins (Rpl and Rps) (FIGS. 12H and 12J). In addition, members of translational elongation factors (Ecf1b2, Eef2, Eif3h, and Ecf1g) were up-regulated in cluster H4 (FIG. 121).

Increase of protein synthesis occurs as a consequence of PERK-mediated responses to ER stress (Han J, et al., ER-stress-induced transcriptional regulation increases protein synthesis leading to cell death. Nat Cell Biol. 2013 May; 15(5):481-90). Indeed, UPR genes and members of ER-associated degradation (ERAD) pathway (Edem2, Sdf211, and Delr3) were up-regulated in cluster H4 (FIG. 12J), suggesting increase of misfolded insulin or secretory proteins in ER of Prmt1BiKO beta cells. Severely distended ER membrane observed in electron microscopic analysis clearly indicated elevation of ER stress in Prmt1BiKO islets (FIG. 12G). Prolonged ER stress leads to ROS generation and oxidative damage to DNA. By immunofluorescence staining, we found increased signal of 8-oxo-dG, an oxidized derivative of deoxyguanosine, in Prmt1BiKO islets (FIG. 12L).

Furthermore, induction of antioxidant mechanisms presented by up-regulation of nuclear-encoded mitochondrial ETC components and ROS scavenger genes (Gpx3, Prdx1, and Prdx4) demonstrated elevated ROS level in Prmt1BiKO islets (FIGS. 12K and 12N). These data indicate that in HFD-fed Prmt1BIKO mouse model, loss of mature identity occurs in subset of beta cells concomitant with increase of ER stress and oxidative stress.

### Example 10: CycloZ Treatment Prevents Beta Cell Dysfunction

Transcriptomic analyses of Prmt1BiKO islets help us understanding molecular changes occurring at different stage of beta cell dysfunction. Type 2 diabetes is a progressive disease, and if predisposing factors are not resolved, beta cells lose their cellular identity and function, and finally undergoes apoptosis. Prmt1 BiKO mice fed HFD for extended period (40 weeks) showed severe beta cell dysfunction (FIG. 12O). Histological analysis of islets showed increased ratio of alpha-to-beta cells in islets (FIG. 12P), which can be observed in human islets during the pathogenesis of T2D.

Considering that elevated proinsulin level and increase of immature insulin secretory granule in islets are early events that can result in beta cell dysfunction in Prmt1BiKO mice, we tried targeted pharmacological intervention to prevent progression of beta cell dysfunction using CycloZ, which is a combination drug of cyclo-His-Pro (CHP) and zinc. CycloZ is reported to chelate zinc ions and enhance zinc absorption (Ref). Zinc is an essential element for tissue homeostasis and particularly important for beta cell function, as it forms hexametric structure with insulin monomers inside secretory granule, which is required for storage and secretion of insulin, and alteration of zinc levels occurs in pathogenesis of diabetes (Yang, Endocrine 2014).

Without binding to any theory, we hypothesized that supplementation of extracellular zinc through CycloZ treatment could promote beta cell function. Although recent study reported novel mechanism of anti-obesity and anti-diabetic effects of CycloZ promoting protein acetylation in the liver and adipose tissue (Jeong et al., Diabetes Meta. J., 2023), its specific effect on beta cell function was not evaluated.

To evaluate the protective effects of CycloZ on beta cell function, we started administration of CycloZ to Prmt1 BiKO mice after tamoxifen-induced deletion of Prmt1 at 8 weeks of age and during HFD feeding (FIG. 13A). Body weight, lean mass and fat mass were not changed by CycloZ treatment (FIGS. 13B, 13L and 13M).

CycloZ treatment did not change glucose tolerance of Prmt1BiKO mice fed standard chow diet (FIG. 13C). However, HFD-induced impairment of glucose tolerance was alleviated by CycloZ treatment (FIG. 13D), although insulin sensitivity was not altered (FIG. 13N). CycloZ treatment increased plasma insulin level and insulin content in whole pancreas (FIGS. 13E and 13H), while plasma proinsulin level and proinsulin content showed non-significant changes (FIGS. 13F and 13I). Plasma proinsulin/insulin ratio was significantly decreased in CycloZ-treated mice (FIG. 13G) and pancreas proinsulin/insulin ratio showed non-significant reduction (FIG. 13J). In line with these results, electron microscopic analysis showed marked increase of dense-core insulin granules in beta cells of CycloZ-treated mice (FIG. 13K). Taken together, these data indicate that CycloZ treatment prevents beta cell dysfunction and improves glucose homeostasis.

### Example 11: CycloZ Treatment Protects Beta Cell from Loss of Identity

To elucidate the effects of CycloZ on protection of beta cell identity, we performed scRNA-seq analysis using islets isolated from HFD-fed Prmt1 BiKO mice at 26 weeks of ages after treatment of vehicle or CycloZ for 18 weeks.

We generated duplicate scRNAseq samples for each group to enhance reproducibility. UMAP projection revealed that transcriptional differences between vehicle and CycloZ-treated mice are prominent in beta cells, and less obvious in other endocrine cell types (FIG. 14J). After isolating beta cells, we identified six beta cell clusters by unbiased clustering (FIG. 14A). Each duplicate showed similar cell clustering patterns in UMAP (FIG. 14B). Beta cells from CycloZ-treated mice was distinguished from those of vehicle-treated mice, although there were some overlaps in cell clusters (FIG. 14B).

Cluster 1 and 2 consists of majority of beta cell population in CycloZ-treated islets (FIG. 14K). In cluster 1 and 2, beta cell maturity markers (Mafa) and insulin secretion (Slc2a2 and Slc30a8) were up-regulated and markers for beta cell immaturity and dedifferentiation (Aldh1a3 and Cck) was down-regulated (FIGS. 14C, 14E, and 14L).

Immunofluorescence staining of maturity marker genes confirmed the effects of CycloZ on protection of beta cell identity (FIG. 14D). However, expression of Pcsk2 and Scg5, which were down-regulated by Prmt1 deletion, were not changed in beta cells of CycloZ-treated mice (FIG. 14M), suggesting that proinsulin processing was not normalized by CycloZ treatment. Given that the number of dense core insulin granule was significantly increased in CycloZ-treated mice (FIG. 13J), we hypothesized that zinc supplementation by CycloZ treatment might have beneficial effect on insulin granule formation, as zinc is an essential factor for insulin crystallization in dense core granule (F. C. Schuit, PNAS 2009). We compared zinc content in islets through dithizone staining and observed increased level of zinc in CycloZ-treated islets (FIG. 14N). However, although extracellular zinc level can be increased by supplementation of zinc by CycloZ treatment, zinc influx into the insulin granule is governed by expression of zinc transporters, which was demonstrated by the loss of dense core granules in the beta cells of Slc30a8 knockout mice (F. C. Schuit, PNAS 2009). We observed up-regulation of Slc30a8 in scRNA-seq analysis and immunofluorescence staining of pancreas section of CycloZ-treated mice (FIGS. 14G and 14O). We further explored the expression of genes that can affect intracellular zinc level and found up-regulation of Mt1 and Mt2 genes in CycloZ-treated beta cells (FIG. 14J). Mt1 and Mt2 encodes metallothionein (MT) proteins that act as a reservoir of zinc ion and regulate intracellular zinc homeostasis. These data indicate that CycloZ-induced up-regulation of Slc30a8 and MT genes increase zinc influx into insulin granules and formation of dense-core insulin granules.

Given that elevated proinsulin in Prmt1BiKO beta cell caused ER stress, we explored the effects of CycloZ on the alleviation of ER stress. GSEA analysis revealed that genes involved in UPR, protein modification (Fkbp11, Ssr4, and Creld2) and ERAD pathway (Derl3, Sdf211, and Edem2) were down-regulated in CycloZ-treated beta cell (FIGS. 14F and 14J), suggesting that CycloZ treatment relieved ER-stress generated in HFD-fed Prmt1BiKO islets. Reduction of ER stress was exhibited by the morphology of ER in beta cell of CycloZ-treated mice in comparison with severely distended ER in beta cell of vehicle-treated mice (FIG. 14I). Subsequent reduction of oxidative stress was demonstrated by down-regulation of OXPHOS genes and ROS scavengers (Gpx3, Prdx1, and Prdx4) (FIGS. 14F and 14J), and reduction of 8-oxo-dG staining in islets (FIGS. 14K and 14L). These data showed that CycloZ treatment can relieve ER stress and prevent oxidative stress, thus protecting beta cells from beta cell dysfunction.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as, an acknowledgement or admission or any form of suggestion that that prior publication (or information derived from it) or known matter forms part of the common general knowledge in the field of endeavor to which this specification relates.

What we claim:

1. A method for treating pancreatic beta cells dysfunction or protecting pancreatic beta cells in a subject in need thereof, comprising administering to the subject an effective amount of cyclo (His-Pro) (CHP), a pharmaceutically acceptable salt thereof, stereoisomer, or a solvate thereof.

2. The method of claim 1, wherein the subject is prediabetic, pregnant, or lactating.

3. The method of claim 1, wherein the subject has decreased insulin secreting β-cell mass by at least 10%, at least 20%, at least 30%, at least 40%, at least 50%, at least 60%, at least 70%, at least 80%, at least 90%, compared to a healthy subject.

4. The method of claim 1, further comprising administering the zinc; or administering an antidiabetic agent that is not CHP; or administering the zinc and an antidiabetic agent that is not CHP to the subject.

5. The method of claim 1, wherein the CHP, a pharmaceutically acceptable salt thereof, stereoisomer, or a solvate thereof is administered in a form of composition selected from the group consisting of a nutritional product, a food product, a functional food product, a healthy ageing product, a nutritional supplement, a pharmaceutical composition, a beverage product, and a pet food product.

6. The method of claim 1, wherein the administering to the subject increases expression of MAFA gene, UCN3 gene, SLC2A2 gene, or a combination thereof in beta cells of the subject.

7. The method of claim 1, wherein the administering to the subject suppresses expression of 8-OXO-dG in β-cells of the subject.

8. The method of claim 1, wherein the administering to the subject lowers level of proinsulin in blood and/or in beta cells of the subject; and/or wherein the administering to the subject lowers plasma proinsulin/insulin ratio of the subject.

9. A method for lowering level of proinsulin in blood and/or in beta cells of the subject, and/or lowering plasma proinsulin/insulin ratio in a subject in need thereof, comprising administering to the subject an effective amount of cyclo (His-Pro) (CHP), a pharmaceutically acceptable salt thereof, stereoisomer, or a solvate thereof.

10. The method of claim 9, wherein the administering to the subject increases expression of MAFA gene, UCN3 gene, SLC2A2 gene, or a combination thereof in beta cells of the subject.

11. The method of claim 9, wherein the administering to the subject suppresses expression of 8-OXO-dG in β-cells of the subject.

12. The method of claim 9, further comprising administering the zinc; or administering other antidiabetic agent; or administering the zinc and other antidiabetic agent to the subject.

13. The method of claim 9, wherein the CHP, a pharmaceutically acceptable salt thereof, stereoisomer, or a solvate thereof is administered in a form of composition selected from the group consisting of a nutritional product, a food product, a functional food product, a healthy ageing product, a nutritional supplement, a pharmaceutical composition, a beverage product, and a pet food product.
